SEQUENCE LISTING (8 sequences)

SEQ ID NO: 1 - DNA, 747 bp, Herpes Simplex Virus 1
atggcccgccgccgccgccatcgcggcccccgccgcccccggccgcccgggcccacgggcgccgtcccaaccgcacagtcccaggtaacctccacgcccaactcggaacccgcggtcaggagcgcgcccgcggccgccccgccgccgccccccgccggtgggcccccgccttcttgttcgctgctgctgcgccagtggctccacgttcccgagtccgcgtccgacgacgacgatgacgacgactggccggacagccccccgcccgagccggcgccagaggcccggcccaccgccgccgccccccggccccggcccccaccgcccggcgtgggcccggggggcggggctgacccctcccaccccccctcgcgccccttccgccttccgccgcgcctcgccctccgcctgcgcgtcaccgcggagcacctggcgcgcctgcgcctgcgacgcgcgggcggggagggggcgccggagccccccgcgacccccgcgacccccgcgacccccgcgacccccgcgacccccgcgcgggtgcgcttctcgccccacgtccgggtgcgccacctggtggtctgggcctcggccgcccgcctggcgcgccgcggctcgtgggcccgcgagcgggccgaccgggctcggttccggcgccgggtggcggaggccgaggcggtcatcgggccgtgcctggggcccgaggcccgtgcccgggccctggcccgcggagccggcccggcgaactcggtctaa

SEQ ID NO: 2 - DNA, 2541 bp, Human cytomegalovirus
atggcccagcgcaacggcatgtcgccgcgccccccgccccttggtcgcggccgcggggccggagggccttcgggggttggttcctctcctccttcttcttgtgtgccgatgggagcgccgtcaacagcgggcactggtgcgagtgctgcggctacgacgacgccgggccacggcgtccaccgggtagaaccccgcgggccgccgggcgcccctccgagtagcggcaacaatagcaacttttggcacggcccggagcgcctgttgctgtctcagattccggtggagcgccaggcgctgacggagctggaataccaggccatgggcgccgtgtggcgcgcggcgtttttggccaacagcacgggccgcgccatgcgcaagtggtcgcagcgcgacgcgggcacgctgctgccgctcggacggccgtacggattctacgcgcgggtgacgccgcgcagccagatgaacggcgtgggcgcgacggacctgcgtcaactgtcgccgcgggacgcgtggatcgtactggtggctaccgtggtgcacgaggtggaccccgcagccgacccgacggtgggcgacaaggccggccatcccgagggtctgtgcgcgcaggacggactgtacctggcgctgggcgccgggttccgcgtgttcgtgtacgacctggcaaacaacacgctgatcctagcggcgcgcgacgcggacgagtggtttcggcacggcgcgggcgaggtggtgcggctgtaccgctgcaaccggctgggcgtgggcaccccgcgcgcgacgctgctgcctcagccggcgctccgacagacgttgctgcgcgccgaggaggcgacggcgctcggacgggagctgcgccggcggtgggccggcacgacggtggcgctgcagacgccgggcaggcgactgcagccgatggtactgctgggcgcgtggcaggagctggcgcagtacgagccgttcgcgtcggcgccgcaccccgcgtcgctgctgacggccgtgcgtcggcacctgaaccagcgtctgtgctgcggctggctggcgctgggcgcggtgctgcccgcgcggtggctgggctgcgcggcggggccggcgacggggacggcggcggggacgacgtcgccgccagcggcgagcggcacggagacggaggccgccggcggggacgcgccgtgcgcgatagcgggagccgtggggtccgctgtacctgtgcctccgcagccgtacggcgccgccggcgggggcgcgatttgcgtgcctaacgcggacgcgcacgcggtggtcggggcggacgcggcagcagcagcggcgccgacggtgatggtgggttcgacagcgatggcgggtccggcggcgtcggggaccgtgccgcgcgccatgctggtggtgctgctggacgagctgggcgccgtgttcgggtactgcccgctggacgggcacgtgtacccgctggcggcggagctgtcgcactttctgcgcgcgggcgtgctgggcgcgctggcgctgggacgcgagtcggcgcccgccgccgaggccgcgcggcggctgctgcccgagctggaccgcgagcagtgggagcggccgcgctgggacgcgctgcacctgcacccgcgcgccgcgctgtgggcgcgcgagccgcacgggcagtgggagttcatgtttcgcgaacaacgcggtgaccccataaatgatcccctcgcatttcgtctttcggacgctcgaactctcggtctcgacctcaccaccgtcatgacagagcgtcaaagtcaattgcccgaaaagtatatcggtttctatcagattaggaaacctccttggctcatggaacaacctccacccccatctcgccaaaccaaaccggacgctgcaacgatgcccccaccgctcagtgctcaggcaagcgtcagctacgcgctccgatacgatgacgagtcctggcgcccgctcagcacagttgacgaccacaaagcctggttggatctcgacgaatcacattgggtcctcggggacagccgacccgacgatataaaacaacgcagactgctgaaggccactcaacgacgaggcgccgaaatcgacagacccatgcctgtcgtgcctgaagaatgttacgaccaacgcttcactaccgaaggccaccaggtcatcccgttgtgcgcgtccgaacccgaggatgacgacgaagatcctacctacgacgaattgccgtcgcgcccaccccagaaacataagccgccagacaaacctccgcgcttatgcaaaacgggccccggcccacctccgctgccgccaaagcaacggcacggttccaccgacggaaaagtttctgcgccccgacagtcggagcatcataaaagacagacccgaccgccaaggccgccaccgcccaaattcggggatagaaccgcggcccatctctcgcaaaatatgcgggacatgtacctcgatatgtgtacatcttcgggccacaggccacggccgccagcacctccgcggccgaaaaaatgtcaaacacacgcccctcaccacgttcatcattga

SEQ ID NO: 3 - DNA, 2367 bp, Human cytomegalovirus
ttattgagcattgtaatggtagtgtgtggctatattagaaaacgtgacgcgtcgcatgtcgcggcacaatctggcagcggggtcggggtagggtacggtgggaggcatgtacacagatggaacaaaagcagaagtaacgtgagaaggagcatacagtccagtatccagcggttcctgagtagcaccacccatcaactgaatgccctcatgagtaaaagtctgcgggcgacagcccttggggaccgttggcatgggacgatcaatctccaaaccacagcgtaacaccgttttcttccaacgtcgttgatagacgtcgtttttacggttactcccaagaacccagaaagtctcgtccaagtcgtaccaggaatcttctccggggagacgcgacggtttccaatcctcgtcgtctcgtctcaaagcacgtcccaaactggcttgaggagtcaacggtggttctgtgggtcgggtgtagcgcgagtgttttcccttcatgagcgattcatcctccttgcctttaggctttttggtctttttgtgtatcatctggccgccggcctccataaccaccgtggccaagtccagtcccagagcttgagcgtcggcgcggcgtcgggcgtcttgcaggtagtcttccacatttgcacagatggccgggtgtttggtggctagggtgaggacctcagcctcgccgcgacccggacgtagcaaaaaagccaactgcccgtgcggctcgcgcgcccacagcgcggcgcgcgggtgcaggtgcagcgcgtcccagcgcggccgctcccactgctcgcggtccagctcgggcagcagccgccgcgcggcctcggcggcgggcgccgactcgcgtcccagcgccagcgcgcccagcacgcccgcgcgcagaaagtgcgacagctccgccgccagcgggtacacgtgcccgtccagcgggcagtacccgaacacggcgcccagctcgtccagcagcaccaccagcatggcgcgcggcacggtccccgacgccgccggacccgccatcgctgtcgaacccaccatcaccgtcggcgccgctgctgctgccgcgtccgccccgaccaccgcgtgcgcgtccgcgttaggcacgcaaatcgcgcccccgccggcggcgccgtacggctgcggaggcacaggtacagcggaccccacggctcccgctatcgcgcacggcgcgtccccgccggcggcctccgtctccgtgccgctcgccgctggcggcgacgtcgtccccgccgccgtccccgtcgccggccccgccgcgcagcccagccaccgcgcgggcagcaccgcgcccagcgccagccagccgcagcacagacgctggttcaggtgccgacgcacggccgtcagcagcgacgcggggtgcggcgccgacgcgaacggctcgtactgcgccagctcctgccacgcgcccagcagtaccatcggctgcagtcgcctgcccggcgtctgcagcgccaccgtcgtgccggcccaccgccggcgcagctcccgtccgagcgccgtcgcctcctcggcgcgcagcaacgtctgtcggagcgccggctgaggcagcagcgtcgcgcgcggggtgcccacgcccagccggttgcagcggtacagccgcaccacctcgcccgcgccgtgccgaaaccactcgtccgcgtcgcgcgccgctaggatcagcgtgttgtttgccaggtcgtacacgaacacgcggaacccggcgcccagcgccaggtacagtccgtcctgcgcgcacagaccctcgggatggccggccttgtcgcccaacgtcgggtcggctgcggggtccacctcgtgcaccacggtagccaccagtacgatccacgcgtcccgcggcgacagttgacgcaggtccgtcgcgcccacgccgttcatctggctgcgcggcgtcacccgcgcgtagaatccgtacggccgtccgagcggcagcagcgtgcccgcgtcgcgctgcgaccacttgcgcatggcgcggcccgtgctgttggccaaaaacgccgcgcgccacacggcgcccatggcctggtattccagctccgtcagcgcctggcgctccaccggaatctgagacagcaacaggcgctccgggccgtgccaaaagttgctattgttgccgctactcggaggggcgcccggcggcccgcggggttctacccggtggacgccgtggcccggcgtcgtcgtagccgcagcactcgcaccagtgcccgctgtggacggcgctcccatcggcacacaagaagaaggaggagaggaaccaacccccgaaggccctccggccccgcggccgcgaccaaggggcggggggcgcggcgacatgccgttgcgctgggccat

SEQ ID NO: 4 - DNA, 390 bp, Human cytomegalovirus
atggcccagcgcaacggcatgtcgccgcgccccccgccccttggtcgcggccgcggggccggagggccttcgggggttggttcctctcctccttcttcttgtgtgccgatgggagcgccgtccacagcgggcactggtgcgagtgctgcggctacgacgacgccgggccacggcgtccaccgggtagaaccccgcgggccgccgggcgcccctccgagtagcggcaacaatagcaacttttggcacggcccggagcgcctgttgctgtctcagattccggtggagcgccaggcgctgacggagctggaataccaggccatgggcgccgtgtggcgcgcggcgtttttggccaacagcacgggccgcgccatgcgcaagtggtcgcagcgc

SEQ ID NO: 5 - DNA, 24 bp, Artificial Sequence
  Primer
ggatcctcaatggcccagcgcaac

SEQ ID NO: 6 - DNA, 25 bp, Artificial Sequence
  Primer
aagcttatgatgaacgtggtgaggg

SEQ ID NO: 7 - DNA, 28 bp, Artificial Sequence
  Primer
ggatccatcatgacagagcgtcaaagtc

SEQ ID NO: 8 - DNA, 26 bp, Artificial Sequence
  Primer
aagcttcaatgatgaacgtggtgagg

(19) United States
(12) Patent Application Publication
(10) Pub. No.: US 2008/0206199 A1
(43) Pub. Date: Aug. 28, 2008

(54) Chimeric Herpes Viruses and Uses Thereof

(75) Inventors: Kevin Cassady, Birmingham, AL (US); James Markert, Birmingham, AL (US)

Correspondence Address: FISH & RICHARDSON P.C., P.O BOX 1022, Minneapolis, MN, 55440-1022, US

(73) Assignee: THE UAB RESEARCH FOUNDATION, Birmingham, AL (US)

(21) Appl. No.: 11/994,158

(22) PCT Filed: Jun. 30, 2006
(86) PCT No.: PCT/US2006/026052

§ 371 Date: Dec. 28, 2007

Related U.S. Application Data
(60) Provisional application No. 60/696,003, filed on Jul. 1, 2005.
(60) Provisional application No. 60/729,707, filed on Oct. 24, 2005.

(51) Int. Cl. A61K 35/76; C12N 7/01; C12N 15/00; A61P 35/04; C12N 5/02

(52) U.S. Cl. 424/93.2; 435/235.1; 435/375; 435/320.1; 435/440

(57) ABSTRACT

Disclosed herein are chimeric herpesviruses as well as methods of making and using such chimeric herpesviruses. The chimeric viruses comprise two nucleic acid sequences, one from a herpesvirus and one from a different virus. The herpesvirus nucleic acid sequence is a modified protein kinase R (PKR) evasion gene. The second viral nucleic acid sequence inhibits PKR-mediate protein shutoff in tumor cells, but is not neurovirulent. Thus, the chimeric virus has reduced neurovirulence as compared to the wild-type herpesvirus but remains replication competent.

### CROSS-REFERENCE TO PRIORITY APPLICATIONS

This application claims priority to U.S. Provisional Application No. 60/696,003, filed Jul. 1, 2005 and U.S. Provisional Application No. 60/729,707 filed Oct. 24, 2005. The applications to which the present application claims benefit are herein incorporated by reference in their entirety.

### STATEMENT REGARDING FEDERALLY SPONSORED RESEARCH

This invention was made with government support under Grant NCI P01 CA 5 71933 awarded by the National Cancer Institute/HHS/PHS/NIH. The government has certain rights in the invention.

### BACKGROUND

Glioblastoma multiforme (GBM), the most common primary brain tumor, has proven to be one of the most refractory and fatal cancers, conferring a dismal prognosis from the lack of efficacy of traditional therapies including surgery, chemotherapy and radiotherapy. The median life expectancy for patients diagnosed with a GBM is approximately 12 to 15 months, and less than 5% will survive to five years past the date of their diagnosis. The recalcitrant nature of these malignant tumors has resisted new developments in the traditional therapies over the past fifty years and has stimulated intensive investigation into alternative treatment modalities, particularly microbiological targeting of tumors. One promising therapeutic technique currently being explored is the use of herpes simplex virus-1 (HSV-1) vectors to treat cancers such as, for example, breast cancer, gliomas, prostate cancer, lung cancer, colorectal cancer, liver cancer, and head and neck squamous cell carcinoma. Problems with the therapy exist, however, because of the innate host antiviral response.

Herpes simplex viruses are large, enveloped, DNA viruses with a genome of approximately 152 kilobase (kb) pairs. Genetically modified HSV are attractive as replication-competent, oncolytic vectors for a number of reasons. For example, multiple genes can be deleted and/or replaced with therapeutic foreign genes without affecting the replication capacity of the virus and modified herpesviruses can be engineered to retain sensitivity to standard antiviral drug therapy as a “built-in” safety feature (Cobbs et al. 1999; Markert et al. 2000; Rampling et al. 2000).

One of the mechanisms by which a cell responds to viral infection and replication is activation of double-stranded RNA activated protein kinase (PKR). This evolutionarily conserved, interferon-inducible enzyme is present at low levels in a non-active form in unstressed cells, but is induced by interferon or double-stranded RNA (dsRNA) produced during viral infection. The activated enzyme phosphorylates the α subunit of translation initiation factor 2 (eIF-2α) and inhibits protein synthesis initiation in the infected cell. This innate antiviral response to infection limits viral growth during the initial phases of viral infection prior to recruitment of the adaptive immune response. Viruses have evolved a number of ways to block the effect of activated PKR and the genes in several herpesvirus genomes that carry out this function have been identified.

In the case of HSV-1, the γ₁34.5 gene is the principal viral defense against an innate host antiviral response and encodes a multifunctional protein with at least two independent functions. One is the maintenance of late viral protein synthesis in infected cells and is encoded by the carboxyl-terminal domain of the γ₁34.5 gene (Bin He, 1996). During the process of infection, wild-type HSV-1 produces complementary mRNA transcripts that anneal, forming stable double stranded RNA (dsRNA), triggering the dimerization and activation of dsRNA-activated host protein kinase R (PKR). Activated PKR then phosphorylates and inactivates the α subunit of eukaryotic initiation factor (eIF-2α), a rate limiting component of the initiation complex that in its activated form allows methionine incorporation during peptide synthesis. This selective inactivation of eIF-2α in effect leads to the cessation of protein synthesis in the infected cell. The HSV-1 γ₁34.5 protein (ICP34.5) overcomes this PKR-mediated host protein shutoff by binding and recruiting a host phosphatase that specifically dephosphorylates eIF-2α, allowing continued viral protein synthesis, or the wild-type protein synthesis phenotype, in the infected cell (Bin, 1995). Recombinant viruses that lack the γ₁34.5 gene (Δγ₁34.5 recombinants) are incapable of maintaining eIF 2α in an unphosphorylated form and are therefore unable to maintain protein synthesis in the infected cell (Chou, 1992). The cessation of protein synthesis in the infected cell is seen at the onset of viral DNA synthesis late in infection (˜6 to 8 hours post-infection (hpi)) and is referred to as host-mediated protein shutoff, or the Δγ₁34.5 phenotype. The Δγ₁34.5 recombinant viruses described previously consequently replicate inefficiently and generate lower amounts of progeny virus in cells with intact PKR pathways (Cassady, 2002).

A second function encoded by the γ₁34.5 gene grants the virus the ability to efficiently replicate in post-mitotic neuronal cells, conferring a neurovirulent phenotype to HSV-1 (Chou, 1991). Previous studies have shown that this function is independent of the protein shutoff phenotype (He, 1997). Recombinant Δγ₁34.5 viruses described previously, are therefore incapable of efficient replication after direct inoculation in the central nervous system and do not produce encephalitis (Chou, 1990). Deletion of the γ₁34.5 gene renders the virus safe for direct inoculation into the central nervous system (CNS) tumors but also eliminates efficient viral replication in the tumor by inhibiting late virus gene expression. These functions, while encoded by a single gene in HSV-1, are independent phenotypes. Additional problems with Δγ₁34.5 viral therapy include a propensity for secondary mutations that may restore viral protein synthesis and replication but also neurovirulence.

The present application addresses the problem of reducing neurovirulence in a herpesvirus without a concomitant loss of protein synthesis and replication competence.

### SUMMARY

Chimeric viruses comprising two nucleic acid sequences, one from a herpesvirus and one from a different virus are described. The herpesvirus nucleic acid sequence is a protein kinase R (PKR) evasion gene modified to reduce the expression or activity of the gene as compared to expression or activity of the evasion gene in the absence of the modification. The second viral nucleic acid sequence restores or compensates for one function of the PKR evasion gene by allowing continued viral protein synthesis and replication. However, the second viral nucleic acid sequence does not restore the other function of the PKR evasion gene, neurovirulence. Thus, the chimeric virus has reduced neurovirulence as compared to the wild-type herpesvirus but remains replication competent.

Also described are methods of making and using the chimeric virus. For example, a method of selectively killing one or more target cells using the chimeric virus is provided. Such methods are useful in vivo for treatment of diseases, including cancer. The methods are also useful in treating recalcitrant tumors, including those of the central nervous system (CNS). The therapeutic use of the chimeric virus can be combined with other treatment modalities, including for example chemotherapeutics or radiation therapy.

Also provided herein is a viral vector comprising the chimeric virus and an exogenous gene of interest. Such vectors are useful for delivering a gene of interest to a target cell.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic representation of the DNA sequence arrangement of the wild-type HSV-1 and HCMV genome and exemplary recombinant HSV-1 viruses. Both HCMV (line 2) and HSV-1 (line 6) have group E genomes characterized by two covalently linked components, L and S, each composed of unique sequences (UL and US) flanked by inverted repeat sequences. The locations of the HCMV IRS1, IRS263, and TRS1 genes in the wild-type HCMV genome are shown (line 1). The HCMV IE2 gene and the location of the in-frame deletion mutation in IE2 86 exon 5 (Δ) is also shown (line 3). Line 4 demonstrates the location of one of the two copies of the HSV-1 γ₁34.5 gene. In the Δγ₁34.5 parent virus, R3616, both copies of the γ₁34.5 gene have been deleted, as represented in line 5. Line 7 represents the UL3, UL4 genetic domain in the wild-type and R3616 genome. Line 9 shows the UL3, UL4 domain of the recombinant virus C101, derived from R3616, containing the CMV immediate early promoter-driven EGFP gene. The recombinant virus C130, represented in line 11, contains the HCMV TRS1 gene under control of the HCMV IE promoter in the UL3, UL4 intergenic region of a Δγ₁34.5 virus. The Δγ₁34.5 recombinant C132 (expressing IRS1 transcript but not IRS1 protein) and C134 are represented in line 13 and 15, respectively. They contain the CMV IE promoter and HCMV IRS1 gene in the UL3, UL4 intergenic region. Lines 8, 10, 12, 14, and 16 represent the predicted fragments produced by PstI restriction digestion of the viral DNAs. The repair viruses C131 and C135 are not included but are predicted to be identical schematically to the C101 virus (line 9). P, PstI.

FIGS. 2A, 2B and 2C shows replication of chimeric C130 and C134 viruses in U251, U87, and D54 cells in vitro.

FIG. 3 shows levels of parent C101 and chimeric C134 viral replication in the presence of exogenous IFNα.

FIGS. 4A, 4B, 4C and 4D show anti-tumor efficacy of chimeric viruses in a human xenograft model of malignant glioma. SCID mice were implanted with 1×10⁶ U87 malignant glioma cells and treated seven days later after randomization into different groups with various doses of either the parent C101 virus, the chimeric C130 or C134 viruses, or saline. FIGS. 4A, 4B, 4C and 4D are graphs of two separate survival studies combined. For clarity the combined survival studies were split to show the efficacy of the “low dose” (5×10⁴ plaque forming units (pfu)) chimerics separately in FIG. 4D.

FIG. 5 shows anti-tumor efficacy of chimeric viruses in A/J bearing the syngeneic murine neuroblastoma N2A brain tumors.

FIG. 6 shows acyclovir-susceptibility of the chimeric C130 and C134 viruses.

FIG. 7 is a schematic of viruses used to construct a chimeric HSV IL-12 recombinant and repair virus.

FIG. 8 is a graph showing tumor volume size after chimeric HSV (C130 2×10⁵), Δγ₁34.5 (R3616 2×10⁵) virus or saline treatment.

### DETAILED DESCRIPTION

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting. Note the headings used herein are for organizational purposes only and are not meant to limit the description provided herein or the claims attached hereto.

### I. Viruses and Viral Vectors

The goal of oncolytic viral therapy is to achieve maximum tumor cell killing while retaining safety in surrounding normal tissue. To achieve this goal, engineered viruses must be able to selectively replicate and spread throughout the tumor bed without affecting adjacent normal tissue. While the Δγ₁34.5 recombinants are safe for intracranial administration, these vectors are severely limited in their replication in tumors (Markert 2000). To improve Δγ₁34.5 based therapy, modifications of the virus are described herein which improve viral replication, spread within the tumor bed and enhance bystander damage to uninfected tumor cells.

Disclosed herein are chimeric herpesviruses as well as methods of making and using the chimeric viruses. The method and compositions may be understood more readily by reference to the following detailed description of particular embodiments and the Examples included therein and to the Figures and their previous and following description.

Genetically modified HSV are attractive as oncolytic vectors for a number of reasons: 1) procedures for constructing recombinant HSV are well established; 2) multiple genes can be deleted and/or replaced with therapeutic foreign genes without affecting the replication capacity of the virus; 3) considerable experience with the biology of HSV and its behavior in humans and nonhuman primates exists in the literature; and 4) modified herpesviruses can be engineered to retain sensitivity to standard antiviral drug therapy as a “built-in” safety feature. Furthermore, HSV genome size, 152 kb, allows transfer of genes 30 kb or more in size.

There are more than 120 animal herpesviruses. All herpesviruses are divided into three subsets: the alpha (α), beta (β) and gamma (γ) herpesviruses. There are 8 human herpesviruses, which are split between the three subsets. Examples of herpesviruses and their corresponding accession numbers are provided in Table 1.

TABLE 1
Herpesviruses

| | | | | NCBI |
|---|---|---|---|---|
| Sub- | | Classi- | | Sequence |
| group | Common Name | fication | Disease | Accession # |
| Alpha | Herpes Simplex | Human | Oral Lesions | NC_001806.1 |
| | Virus 1 (HSV-1) | Herpes- | | GI:9629378 |
| | | virus 1 | | |
| | HSV-2 | HHV-2 | Genital Lesions | NC_001798.1 |
| | | | | GI:9629267 |
| | Varicella Zoster | HHV-3 | Chickenpox | NC_001348.1 |
| | Virus (VZV) | | | GI:9625875 |
| Beta | Human | HHV-5 | Systemic | NC_001347.2 |
| | Cytomegalovirus | | infections, | GI:28373214 |
| | (HCMV or | | blindness and | NC_006273.1 |
| | CMV) | | brain damage in a | GI:52139181 |
| | | | small percentage | |
| | | | of newborns and | |
| | | | in adult immune | |
| | | | compromised. | |
| | Human | HHV-6 | Roseola Infantum | NC_001664.1 |
| | Herpesvirus 6 | | | GI:9628290 |
| | | | | NC_000898.1 |
| | | | | GI:9633069 |
| | Human | HHV-7 | Roseola Infantum | NC_001716.2 |
| | Herpesvirus 7 | | | GI:51874225 |
| Gamma | Epstein Barr | HHV-4 | Infectious | NC_001345.1 |
| | Virus (EBV) | | Mononucleosis | GI:9625578 |
| | | | | NC_007605 |
| | | | | GI:82503188 |
| | Gamma | HHV-8 | Kaposis's | NC_003409.1 |
| | Kaposis's | | Sarcoma, in | GI:18845965 |
| | Sarcoma | | patients with | |
| | Herpesvirus | | AIDS | |

One promising therapeutic technique is the use of herpes simplex virus-1 (HSV-1) vectors to attack cancer cells. Selective replication of these HSV recombinants in tumors can be achieved by deletion of the viral neurovirulence gene, γ₁34.5. Deletion of the HSV-1 neurovirulence gene allows the safe administration of these oncolytic viruses to mitotically active CNS tumors. Although Δγ₁34.5 viruses are capable of entry into non-dividing normal cells in the CNS, these viruses cannot replicate efficiently except in actively dividing cells such as tumor cells (Chour 1990). Therefore such viruses are tumor-selective viruses. Δγ₁34.5 viruses have shown significant efficacy for therapy of brain malignancies in preclinical animal models, and have been demonstrated to be safe in Phase I and II trails in both the U.S., and Great Britain (Markert 2000 and Rampling 2000). The virus examined in the U.S. Trial, G207, contains an additional mutation in the viral ribonucleotide reductase gene U_{L}39 as an additional safety feature to limit viral growth (Shah 2003). However, attenuated HSV-1 (Δγ₁34.5) recombinants are unable to efficiently synthesize viral proteins and this limits viral replication (Chou et al. 1990; Chou et al. 1995; Mohr et al. 1996; Andreansky et al. 1997; Shah et al. 2003). Despite their inefficient replication, oncolytic HSV improve survival in in vivo tumor studies. However, Δγ₁34.5 viruses are unable to consistently eliminate the entire tumor.

Chimeric viruses comprising a modified herpesvirus nucleic acid sequence and a second viral nucleic acid sequence are described. The herpesvirus nucleic acid modification causes reduced expression of a protein kinase R (PKR) evasion gene as compared to expression of the evasion gene in the absence of the modification. The second viral sequence encodes a protein that comprises the protein synthesis function of the PKR evasion gene without the neurovirulence function of the gene. Therefore, the chimeric virus has a reduced neurovirulence as compared to a wild-type herpesvirus. Also as disclosed herein, the provided chimeric virus has enhanced protein synthesis and/or replication as compared to existing attenuated herpesviruses, such as, for example, Δγ₁34.5 HSV. The second nucleic acid sequence of the provided chimeric virus enhances protein synthesis or replication as compared to the protein synthesis or replication of the chimeric virus in the absence of the second viral nucleic acid sequence. The second nucleic acid sequence of the provided chimeric virus can enhance protein synthesis and replication by inhibiting the activation of PKR, inhibiting the phosphorylation of eIF-2α, or enhancing the dephosphorylation of eIF-2α.

Also the provided chimeric virus has enhanced risistance to interferon (IFN) as compared to existing attenuated herpesviruses, such as, for example, Δγ₁34.5 HSV.

The modified herpesvirus nucleic acid can be a modified α herpesvirus virus nucleic acid. Thus, for example, the modified herpesvirus nucleic acid can be a modified HSV-1 nucleic acid or a modified HSV-2 nucleic acid. The modified herpesvirus nucleic acid can also be a β herpesvirus nucleic acid or a γ herpesvirus virus nucleic acid.

Optimally, the PKR evasion gene of the herpesvirus is a γ₁34.5 gene (SEQ ID NO: 1) or homologous gene thereof. Thus, the modification to the herpesvirus nucleic acid sequence can be a modification of a γ₁34.5 gene or homologous gene thereof. The modification to the herpesvirus nucleic acid sequence can also be a modification of a nucleic acid with at least about 70-99% homology, including 70%, 75%, 80%, 85%, 90%, or 95% homology, to the γ₁34.5 gene.

Modifications that can be made to the herpesvirus PKR evasion gene include one or more mutations, deletions, insertions and substitutions. Thus, the modification to the herpesvirus nucleic acid sequence can comprise the complete or partial deletion of a PKR evasion gene such as the γ₁34.5 gene from HSV-1. The modification can comprise an inserted exogenous stop codon or other nucleotide or nucleotides. The modification can comprise the mutation or deletion of the promoter or the insertion of an exogenous promoter that alters expression of the PKR evasion gene. The modification can comprise one or more inserted nucleotides that results in a codon frame-shift. Furthermore, the second viral nucleic acid sequence of the chimera could be substituted for the PKR evasion gene. Optimally, a gene of interest can be substituted for the PKR evasion gene. Methods for making the modifications described herein are well known to those skilled in the art and are described in more detail below.

The second viral nucleic acid sequence of the chimeric virus comprises one phenotype of the PKR evasion gene, protein synthesis and replication in infected tumor cells, but not the other phenotype of the PKR evasion gene, PKR-mediated virulence, e.g., neurovirulence. In other words, the second viral nucleic acid sequence inhibits PKR-mediated protein shutoff without neurovirulence. Thus, the second viral nucleic acid sequence can be any PKR evasion gene or comparable gene that does not cause virulence. The second viral nucleic acid sequence can be derived from homologous viruses. Thus, the second viral nucleic acid sequence of the provided chimeric virus can be an α herpesvirus nucleic acid sequence, P herpesvirus nucleic acid sequence, or γ herpesvirus nucleic acid sequence. Thus, the viral nucleic acid sequence of the provided chimeric virus can be a cytomegalovirus (CMV) nucleic acid sequence.

Examples of suitable nucleic acid sequences that can be used in the provided chimeric virus include, but are not limited to, IRS-1 (SEQ ID NO: 2) and TRS-1 (SEQ ID NO: 3), or homologous genes thereof. The provided chimeric virus can comprise an IRS-1 gene. The provided chimeric virus can also comprise a nucleic acid having at least about 70-99% homology, including about 70%, 75%, 80%, 85%, 90%, 95% homology to the IRS-1 gene. The provided chimeric virus can comprise a TRS-1 gene, or homologous genes thereof. The provided chimeric virus can also comprises a nucleic acid having at least about 70-99% homology, including about 70%, 75%, 80%, 85%, 90%, 95% homology, to the TRS-1 gene.

HCMV IRS1 and TRS1 proteins have a shared 130 amino acid (aa) region that independently interacts with two eukaryotic genes, Nedd4 and TSG101, involved in vesicular transport and lysosomal sorting in the cell. As described in the examples below a chimeric virus comprising either TRS1 or IRS1 have a similar protein synthesis phenotype. Thus, the provided chimeric virus can comprise the nucleic acid sequence that corresponds to the shared 130 aa region of IRS1 and TRS1 (SEQ ID NO: 4). The provided chimeric virus can also comprise a nucleic acid having at least about 70-99% homology, including about 70%, 75%, 80%, 85%, 90%, 95% homology to SEQ ID NO:4.

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman, Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989, which are herein incorporated by reference for at least the material related to nucleic acid alignment. It is understood that any of the methods typically can be used and that, in certain instances, the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method, even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of the calculation methods, although, in practice, the different calculation methods will often result in different calculated homology percentages.

The disclosed nucleic acids may contain, for example, nucleotide analogs or nucleotide substitutes. Non-limiting examples of these and other molecules are discussed herein. It is understood that, for example, when a vector is expressed in a cell, the expressed mRNA will typically be made up of A, C, G, and U.

A nucleotide analog is a nucleotide which contains some type of modification

either the base, sugar, or phosphate moieties. Modifications to nucleotides are well known in the art and include for example, 5-methylcytosine (5-me-C), 5-hydroxymethyl cytosine, xanthine, hypoxanthine, and 2-aminoadenine.

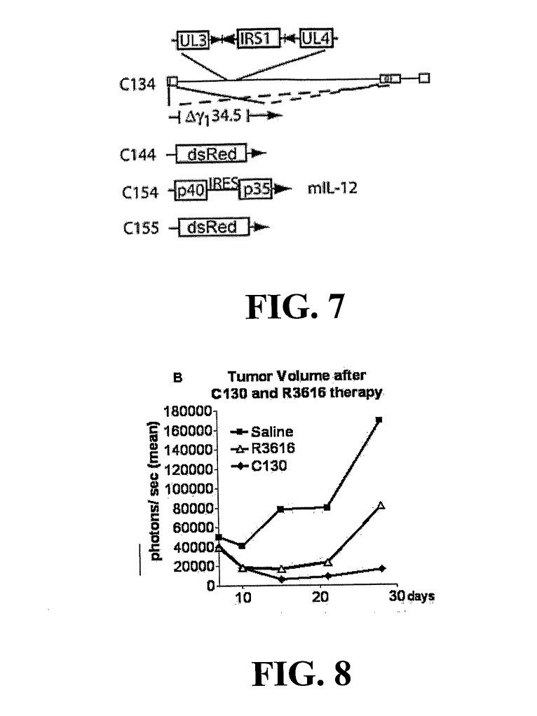

Also provided herein is a viral vector comprising the herein disclosed chimeric virus, wherein the chimeric virus further comprises an exogenous gene of interest. In one embodiment, the gene of interest encodes a therapeutic agent. Since in one embodiment, the disclosed chimeric virus can be used to treat cancer, as discussed below, the therapeutic agent can be a chemotherapeutic agent. As a non-limiting example, the gene of interest can encode HIV-1 GAG. The gene of interest can be an immunomodulatory gene. Suitable immunomodulatory genes include, but are not limited to, IL-12, GM-CSF, IL-15, CCL2, IL-18, IL-24, IL-4, IL-10 and TNF-α. In a preferred embodiment, the gene of interest encodes IL-12. An exemplary viral vector comprising Δγ₁34.5, IRS1 and IL-12 is shown in FIG. 7. It has been shown that Δγ₁34.5 viruses expressing interleukin 12 prolonged survival of immunocompetent mice in an experimental intracranial murine model of neuroblastoma (Parker, et al., 2000). The gene of interest can also be a prodrug converting enzyme such as purine nucleoside phosphorylase (PNP) and cytosine deaminase (CD). The gene of interest can be a viral antigen, such as a non-HSV-1 antigen. Thus, the gene of interest can be an HIV, HSV-2, HCMV, or HHV8 antigen. The gene of interest can also be a tumor-specific antigen.

The gene of interest can also encode a targeting moiety or a marker. In one embodiment, the gene of interest is inserted into the chimeric virus at the γ₁34.5 locus.

Thus, provided is a method of delivering a gene of interest to a cell, comprising contacting the target cell with the herein provided viral vector. The delivery can be in vivo or in vitro.

The chimeric virus of a viral vector optionally comprises a gene encoding a modified HSV glycoprotein required for virus entry. Recombinant HSV have been constructed that exclusively enter tumor cells through tumor-specific receptors (Zhou 2002; Zhou 2005).

Nucleic acids, such as the ones described herein, that are delivered to cells typically contain expression controlling systems. For example, the inserted genes in viral and retroviral systems usually contain promoters and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Preferred promoters controlling transcription from vectors in mammalian host cells may be obtained from various sources, for example, the genomes of viruses such as polyoma, Simian Virus 40 (SV40), adenovirus, retroviruses, hepatitis-B virus and most preferably cytomegalovirus, or from heterologous mammalian promoters, e.g., beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs (bp) in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, a-fetoprotein and insulin), typically one will use an enhancer from a eukaryotic cell virus for general expression. Preferred examples include, but are not limited to, the SV40 enhancer, the cytomegalovirus early promoter enhancer, the polyoma enhancer, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating drugs.

The promoter region can act as a constitutive promoter to maximize expression of the region of the transcription unit to be transcribed. In certain constructs, the promoter region can be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR. It has been shown that all specific regulatory elements can be cloned and used to construct expression vectors that are selectively expressed in specific cell types such as melanoma cells. For example, the glial fibrillary acidic protein (GFAP) promoter has been used to selectively express genes in cells of glial origin. Such tumor specific promoters can also be incorporated into the chimeric viruses as well as the viral vectors described herein.

Expression vectors used in eukaryotic host cells may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs are well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences, alone or in combination with the above sequences, to improve expression from, or stability of, the construct.

The viral vectors can include a nucleic acid sequence encoding a marker product. This marker product is used to determine if the gene has been delivered to the cell and once delivered is being expressed. Marker genes include, for example, the E. Coli lacZ gene, which encodes β-galactosidase, and green fluorescent protein (GFP). Markers can also be used in imaging techniques. Thus, a chimeric vector that encodes a marker could be used to visualize a cancer cell or tumor. The size of the marked region or the intensity of the marker can be used to evaluate the progression, regression, or cure of cancer, for example.

As used herein a “marker” means any detectable tag that can be attached directly (e.g., a fluorescent molecule integrated into a polypeptide or nucleic acid) or indirectly (e.g., by way of activation or binding to an expressed genetic reporter, including activatable substrates, peptides, receptor fusion proteins, primary antibody, or a secondary antibody with an integrated tag) to the molecule of interest. A “marker” is any tag that can be visualized with imaging methods. The detectable tag can be a radio-opaque substance, radiolabel, a fluorescent label, a light emitting protein, a magnetic label, or microbubbles (air filled bubbles of uniform size that remain in the circulatory system and are detectable by ultrasonography, as described in Ellega et al. Circulation, 108:336-341, 2003, which is herein incorporated in its entirety). The detectable tag can be selected from the group consisting of gamma-emitters, beta-emitters, and alpha-emitters, positron-emitters, X-ray emitters, ultrasound reflectors (microbubbles), and fluorescence-emitters suitable for localization. Suitable fluorescent compounds include fluorescein sodium, fluorescein isothiocyanate, phycoerythrin, Green Fluorescent Protein (GFP), Red Fluorescent Protein (RFP), Texas Red sulfonyl chloride (de Belder & Wik, Carbohydr. Res.44(2):251-57 (1975)), as well as compounds that are fluorescent in the near infrared such as Cy5.5, Cy7, and others. Also included are genetic reporters detectable following administration of radiotracers such as hSSTr2, thymidine kinase (from herpes virus, human mitochondria, or other) and NIS (sodium/iodide symporter). Light emitting proteins include various types of luciferase. Those skilled in the art will know, or will be able to ascertain with no more than routine experimentation, other fluorescent compounds that are suitable for labeling the molecule.

In vivo monitoring can be carried out using, for example, bioluminescence imaging, planar gamma camera imaging, SPECT imaging, light-based imaging, magnetic resonance imaging and spectroscopy, fluorescence imaging (especially in the near infrared), diffuse optical tomography, ultrasonography (including untargeted microbubble contrast, and targeted microbubble contrast), PET imaging, fluorescence correlation spectroscopy, in vivo two-photon microscopy, optical coherence tomography, speckle microscopy, small molecule reporters, nanocrystal labeling and second harmonic imaging. Using the aforementioned imaging technologies, reporter genes under control of various inflammation specific promoters are detected following specific induction.

These technologies can be applied in combination with other imaging technologies. For example, tumor mass monitoring can be accomplished using tumor cells positive for CMV-luciferase. In addition, two luciferase enzymes can be imaged at the same time, for example, using CMV-luciferase (from firefly) and cox2L-luciferase (from Renilla). Other reporters and promoters can be used in conjunction with these examples, some examples of which are disclosed above.

The marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells that are not transformed with the DHFR or TK gene are not be capable of survival in non-supplemented media.

The second category is dominant selection which refers to a selection scheme used in any cell type and does not require the use of a mutant cell line. These schemes typically use a drug to arrest growth of a host cell. Transformed cells express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### II. Methods of Making

The compositions disclosed herein and the compositions necessary to perform the disclosed methods can be made using any method known to those of skill in the art for that particular reagent or compound unless otherwise specifically noted. For example, the nucleic acids can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

The chimeric viruses and viral vectors can be made recombinantly as set forth in the examples or by other methods of making recombinant viruses as described in many standard laboratory manuals, such as Davis et al., Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989). Similar methods are used to introduce a gene of interest in methods of making the viral vector described herein. For example, recombinant viruses can be constructed using homologous recombination after DNA co-transfection. In this example, cells can be co-transfected with at least two different viruses containing the genes of interest and progeny virus plaque can be purified based upon loss of marker expression. Final verification of the correct genetic organization of candidate viruses can be verified by DNA hybridization studies using probes to the nucleic acids as described herein.

The nucleic acid sequences described herein may be obtained using standard cloning and screening techniques, from natural sources such as genomic DNA libraries or can be synthesized using well known and commercially available techniques.

When the nucleic acid sequences are used recombinantly, the nucleic acid sequence may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. The nucleic acid sequence may also contain non-coding 5′ and 3′ sequences, such as transcribed, non-translated sequences, splicing and polyadenylation signals, ribosome binding sites and sequences that stabilize mRNA.

The nucleic acids may be used as hybridization probes for cDNA and genomic DNA or as primers for a nucleic acid amplification (PCR) reaction, to isolate full-length cDNAs and genomic clones encoding polypeptides and to isolate cDNA and genomic clones of other genes (including genes encoding homologs and orthologs from different species) that have a high sequence similarity.

The nucleic acids described herein, including homologs and orthologs from species, may be obtained by a process which comprises the steps of screening an appropriate library (as understood by one of ordinary skill in the art) under stringent hybridization conditions with a labeled probe or a fragment thereof; and isolating full-length cDNA and genomic clones containing said polynucleotide sequence. Such hybridization techniques are well known to the skilled artisan.

Modifications that can be made to the herpesvirus PKR evasion gene include one or more mutations, deletions, insertions and substitutions. Methods for making modifications to nucleic acid sequence are well known to those of skill in the art. As used herein, “modified PKR evasion gene” means that one or more nucleotides are altered, relative to wild-type PKR evasion gene, in one or more regions such that the activity of the modified PKR evasion gene is decreased, preferably absent, relative to wild-type PKR evasion gene. The mutation may be caused in a variety of ways including one or more frame shifts, substitutions, insertions and/or deletions, including nonsense mutations (amber (UAG), ocher (T/UAA) and opal (T/UGA)). The deletion may be of a single nucleotide or more, including deletion of the entire gene.

### IV. Methods of Using

The disclosed methods and compositions are applicable to numerous areas including, but not limited to, use as research tools for the study of cancer cell resistance to viral therapies. Other uses are disclosed, apparent from the disclosure, and/or will be understood by those in the art.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

As disclosed herein, while existing attenuated herpesviruses, such as, for example, Δγ₁34.5 HSV, mediate tumor cell destruction primarily via an autophagic mechanism, the provided chimeric virus inhibits the autophagic response and produces a classical viral lytic cellular death. During the antiviral response, protein synthesis initiation is coupled with the bulk protein degradation in the cell, also called autophagy (Franklin et al. 1998; Talloczy et al. 2002). Viruses have evolved to selectively regulate the cellular responses to infection by targeting different components of the PKR pathway. The γ₁34.5 gene, in addition to regulating protein synthesis initiation, modifies autophagy in the HSV-infected cell (Franklin et al. 1998; Talloczy et al. 2002). The Δγ₁34.5 recombinant induces cellular stress similar to nutrient starvation but is unable to prevent autophagy (Talloczy et al. 2002). Viral inhibition of cellular autophagy provides an advantage to the virus by maintaining cellular viability and preventing the release of lysosomal contents into the cell during replication. Viral control over cytoplasmic vesicular transport is also advantageous for the virus in order to negotiate envelopment and transit out of the cell.

Methods of selectively killing a target cell wherein the cell is contacted with a chimeric virus or viral vector are described. Target cells include, but are not limited to cancer cells. The contracting step can be performed in vitro or in vivo. Thus, in one aspect, the disclosed chimeric virus can be used to treat any disease where uncontrolled cellular proliferation occurs, such as in cancer. The target cell can be a solid tumor cell. The disclosed chimeric virus can also be used to treat a precancer condition such as cervical and anal dysplasia, other dysplasia, severe dysplasia, hyperplasia, atypical hyperplasia, or neoplasia. Thus, the target cell can be a adenocarcinoma, hepatoblastoma, sarcoma, glioma, glioblastoma, neuroblastoma, plasmacytoma, histiocytoma, melanoma, adenoma, myeloma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, or pancreatic cancer. The target cells can be ectodermally-derived cancer cells. The target cells can be brain cancer cells. Thus, the target cell can be a neuroblastoma cell, glioma cell, or glioblastoma cell. The target cell can be a breast cancer cell. The target cell can be a hepatoblastoma cell or liver cancer cell. The method of killing a targeted cell can further comprise additional steps known in the art for promoting cell death.

Also provided herein is a method of treating cancer in a subject comprising contacting a cancer cell with the herein provided chimeric virus. The cancer can be selected from the group consisting of adenocarcinoma, sarcoma, glioma, glioblastoma, neuroblastoma, plasmacytoma, histiocytoma, melanoma, adenoma, myeloma, hepatoblastoma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, and pancreatic cancer. Thus, the cancer can be a glioma. Thus, the cancer can be a glioblastoma. The cancer can be a neuroblastoma. The cancer can be a breast cancer. The cancer can also be pancreatic cancer or hepatoblastoma.

A. Combination Therapies

The provided methods can further comprise administering to the subject a chemotherapeutic agent, including biologicals, radiation therapy, or a combination thereof. Biological therapies are naturally occurring or synthesized substances that direct, facilitate, or enhance the body's normal immune defenses. Biologic therapies include interferons, interleukins, monoclonal antibodies, vaccines, and other compounds. Monoclonal antibodies are proteins that can be made in the laboratory and are designed to recognize and bind to very specific sites on a cell. This binding action promotes anticancer benefits by eliminating the stimulating effects of growth factors and by stimulating the immune system to attack and kill the cancer cells to which the monoclonal antibody is bound. The following are non-limiting examples of biological therapies being tested alone or in combination with chemotherapy in clinical trials: 06-benzylguanine (with bischloroethylnitrosourea (BCNU)); ABT-627; ADVEXIN® (Introgen Therapeutics, Inc., Austin, Tex.) gene therapy; AP23573; Arsenic trioxide; BCNU, Carmustine; BG00001; Bortezomib, PS-341; Carboplatin; CCI-779 (Rapamycin Analog Drug); Celecoxib; Cilengitide; Cisplatin; CMT-3, COL-3, Collagenex; Dalteparin sodium; Edotecarin; ERBITUX® (Imclone Systems Incorporated, New York, N.Y.) (cetuximab); Erlotinib; Etoposide; Gefitinib (ZD1839); GRN163L; HERCEPTIN® (Trastuzumab) (Genentech, Inc., San Francisco, Calif.); Human reovirus; IL13-PE38QQR; Imatinib mesylate (STI571); Irinotecan hydrochloride; J-107088; Lomustine; MLN608; Polifeprosan 20 with carmustine implant; Poly ICLC; Procarbazine; PTK787/ZK-222584; Rsr-13 (efaproxiral sodium); SCH66336; Sirolimus; SU5416 (semaxanib); Talampanel; Tamoxifen; Temozolomide; Thalidomide; Tipifarnib (R115777, FTI); TNT-Tumor necrosis therapy; Vincristine; or VIRULIZIN® (Lorus Therapeutics, Inc., Toronto, Ontario, Canada). The methods described herein include administering such exemplary biological therapies to a subject. In another embodiment, the provided method comprises administering to the subject a mammalian Target Of Rapamycin (mTOR), an enzyme activated through the PI3K/Akt cascade. This blockade leads to cell arrest in G1. Rapamycin and its analogs are cytostatic against xenografts of glioblastoma, medulloblastoma, breast cancer, and prostate cancer.

As an example, the chimeric virus can be administered to a subject in need in combination with ionizing radiation (IR). Intratumor injection of Δγ₁34.5 viruses into U87-MG tumors in nude mice, followed by irradiation, improved survival of mice over either therapy alone (Advani 1998). Other studies have demonstrated that in multiple tumor models, IR improves the replication of a variety of recombinants, including a virus containing a copy of the γ₁34.5 gene (Advani 1999; Chung 2002). Administration of IR typically occurs between 6 to 24 hours after viral dosing. Suitable dosages of IR include, but are not limited to 5 to 20 grays (GY). Improved viral protein synthesis and increased viral replication after external beam IR accounted for at least part of the mechanism of the increased tumor-specific killing (Mezhir 2005; Smith 2006). No increased toxicity was noted with this combined treatment.

The chimeric virus can also be administered to a subject in need in combination with temozolomide (TMZ) an oral alkylating agent that is approved for treatment of GBM. Combinatorial TMZ and G207 oncolytic HSV, described above, therapy has been shown to improve survival over either therapy alone in animal studies.

### VI. Formulations and Methods of Administration

The herein provided chimeric viruses and viral vectors can be administered in vitro or in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Imnunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. For review, see Brown and Greene, DNA and Cell Biology 10:6, 399-409 (1991).

Pharmaceutical compositions may include carriers, thickeners, diluents, buffers, preservatives, surface active agents and the like in addition to the molecule, in this case virus or viral vector, of choice. Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of a pharmaceutically-acceptable carriers include, but are not limited to, saline, Ringer's solution and dextrose solution. The pH of the solution is preferably from about 5 to about 8, and more preferably from about 7 to about 7.5. Further carriers may include sustained release preparations such as semipermeable matrices of solid hydrophobic polymers containing the antibody, which matrices are in the form of shaped articles, e.g., films, liposomes or microparticles. It will be apparent to those skilled in the art that certain carriers may be more preferable depending upon, for instance, the route of administration and concentration of composition being administered.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as; for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The viruses and vectors can be administered in a number of ways depending on whether local or systemic treatment is desired, and on the area to be treated. Administration may be topical, oral, by inhalation, or parenterally, for example by intravenous drip, subcutaneous, intraperitoneal or intramuscular injection. The disclosed viruses and vectors can be administered intravenously, intraperitoneally, intramuscularly, subcutaneously, intracavity, or transdermally. Thus, administration of the provided viruses and vectors to the brain can be intracranial, subdural, epidural, or intra-cisternal. For example, the provided viruses and vectors can be administered directly into the tumors by stereotactic delivery. It is also understood that delivery to tumors of the CNS can be by intravascular delivery if the virus or vector is combined with a moiety that allows for crossing of the blood brain barrier and survival in the blood. Thus, agents can be combined that increase the permeability of the blood brain barrier. Agents include, for example, elastase and lipopolysaccharides. The provided viruses and vectors are administered via the carotid artery. In another aspect, the provided viruses and vectors are administered in liposomes, such as those known in the art or described herein. The provided viruses and vectors can be administered to cancers not in the brain intravascularly or by direct injection into the tumor.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein for the methods taught therein.

It is also possible to link molecules (conjugates) to viruses or viral vectors to enhance, for example, cellular uptake. Conjugates can be chemically linked to the virus or viral vector. Such conjugates include but are not limited to lipid moieties such as a cholesterol moiety. (Letsinger et al., Proc. Natl. Acad. Sci. USA, 1989, 86:6553-6556).

The viruses and viral vectors described herein may be administered, for example, by convection enhanced delivery, which has been used with adenovirus and AAV to increase the distribution of the virus thorough bulk flow in the tumor interstitium (Chen 2005). Genetic modifications have also been used to enhance viral spread. For example, insertion of the fusogenic glycoprotein gene produced an oncolytic virus with enhanced antiglioma effect (Fu 2003). Therefore, the viral vectors described herein may comprise such a gene.

A. Dosages

The exact amount of the compositions required will vary from subject to subject, depending on the species, age, weight and general condition of the subject, the severity of the disease being treated, the particular virus or vector used, its mode of administration and the like. Thus, it is not possible to specify an exact amount for every composition. However, an appropriate amount can be determined by one of ordinary skill in the art using only routine experimentation given the teachings herein.

Effective dosages and schedules for administering the compositions may be determined empirically, and making such determinations is within the skill in the art. For example, there are several brain tumor models that provide a mechanism for rapid screening and evaluation of potential toxicities and efficacies of experimental therapies. There are six separate human glioma xenograft models used for critical studies (Pandita 2004). There is also available a spontaneously derived syngeneic glioma model that does not express foreign antigens commonly associated with chemically or virally induced experimental tumors (Hellums 2005). Other animals models for a variety of cancers can be obtained, for example, from The Jackson Laboratory, 600 Main Street Bar Harbor, Me. 04609 USA, which provides hundreds of cancer mouse models. Both direct (histology) and functional measurements (survival) of tumor volume can be used to monitor response to oncolytic therapy. These methods involve the sacrifice of representative animals to evaluate the population, increasing the animal numbers necessary for the experiments. Measurement of luciferase activity in the tumor provides an alternative method to evaluate tumor volume without animal sacrifice and allowing longitudinal population-based analysis of therapy.

The dosage ranges for the administration of the compositions are those large enough to produce the desired effect in which the symptoms of the disease are affected. The dosage should not be so large as to cause adverse side effects, such as unwanted cross-reactions, anaphylactic reactions, and the like. The dosage can be adjusted by the individual physician in the event of any counterindications. Dosage can vary, and can be administered in one or more dose administrations daily, for one or several days.

Viral recovery and immunohistochemistry have been used successfully to monitor viral replication and spread in vivo. Bioluminescent and fluorescent protein expression by the virus can also be used to indirectly monitor viral replication and spread in the tumor. Genes encoding fluorescent reporter proteins (d2EGFP and dsRED monomer) or bioluminescent markers (firefly luciferase) are commonly used in recombinant viruses. Not only do these facilitate the screening and selection of recombinant viruses in vitro. The reporter genes also allow indirect monitoring of viral activity in the in vivo studies.

The provided chimeric viruses require lower dosing as compared to existing attenuated herpesviruses. The provided chimeric virus significantly improves survival as compared to conventional attenuated herpesviruses, such as, for example, Δγ₁34.5 HSV, and is effective at lower doses. For example, the disclosed chimeric virus is effective at from about 10³ pfa, including 10⁴, 10⁵, 10⁶, 10⁷, 10⁸, and 10⁹ pfu, or any amount in between. Thus, the dose of chimeric virus can be from 5×10³ to 5×10⁶ pfu, more preferably from 5×10⁴ to 5×10⁵.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for.

### Example 1

Human CMV TRS1 and IRS1 Gene Products Block the dsRNA Activated Host Protein Shutoff Response Induced by Herpes Simplex Virus Type 1 Infection

(i) Materials and Methods

Cells, viruses, and plasmids. Vero cells were obtained from the American Type Tissue Culture Collection 10801 University Blvd., Manassas, Va. 20110 and were propagated in Dulbecco's modified Eagle medium (DMEM) supplemented with 5% newborn calf serum (Cassady, K. A., et al. 1998a). Primary HFF cells were prepared as previously described and maintained for a maximum of 10 passages in 10% fetal bovine serum (FBS) (Williams, S. L., et al. 2003). The mammalian expression plasmids pHCMV 214, pHCMV 215, and pHCMV 231, encoding the polyhistidine-tagged IRS1, TRS1, and IRS263 protein coding domains, respectively, have been described previously (Romanowski, M. J., et al. 1997). The plasmid pCK1029 was created by inserting a 1.3-kb fragment, encoding two 8-base PacI restriction sites flanking the HCMV IE promoter and the coding domain of enhanced green fluorescent protein (EGFP) (Clontech, Palo Alto, Calif.), into the ApoI site in the UL3, UL4 intergenic region of plasmid pRB4841. The plasmids pCK1114 and pCK1116 contain the SpeI fragment from the mammalian expression plasmids pHCMV215 and pHCMV214, respectively, inserted in the HSV-1 UL3, UL4 intergenic region. The plasmid pCK3008 was constructed by inserting the IRS1 gene in frame with the carboxyl-terminal epitope and polyhistidine tag AD-169 viral DNA using Pfu polymerase and the 5′ BamHI TRS1 (5′-GGATCCTCAATGGCCCAGCGCAAC-3′) (SEQ ID NO: 5) and 3′ HindIII IRS263 (5′-AAGCTTATGATGAACGTGGTGAGGG-3′) (SEQ ID NO: 6) oligonucleotides. The amplified DNA was then incubated with Taq polymerase, gel isolated, and cloned into pcDNA3.1/V5-His-TOPO (Invitrogen, Carlsbad, Calif.). The clone was verified by restriction digestion analysis and detection of immunoreactive protein of the expected mass, using IRS1 antisera in transient expression assays. A 4.4-kb BglII, AvrII fragment encoding the HCMV immediate early (IE) promoter and epitope-tagged IRS1 gene product from pCK3008 was then inserted into the UL3/4 intergenic region, thus creating the pCK1127 clone. HSV-1 (F) and AD169 are the prototypical HSV-1 and HCMV strains, respectively, used in these experiments (Ejercito, P. M., et al. 1968; Pritchett, R. F. 1980). The HSV-1 recombinant virus, R3616, lacks 1,000 bp in both copies of the γ₁34.5 gene (Chou, J., et al. 1990). The γ₁34.5 gene product is the principal defense against PKR mediated host protein shutoff, and the Δγ₁34.5 virus R3616 triggers the host protein shutoff response in human cells (Chou, J. and B. Roizman. 1992). The recombinant herpesviruses in this study were created by co-transfection and homologous recombination as previously described (Post, L. E. and B. Roizman. 1981). C101 is a Δγ₁34.5 HSV-1 recombinant that expresses EGFP. It was isolated from among the progeny created by co-transfection of the plasmid pCK1029 and R3616 DNA and was purified with Vero cells by EGFP-positive plaque selection. The Δγ₁34.5 HSV-1 recombinant C130, which expresses the HCMV TRS1 gene product, was isolated in Vero cells on the basis of loss of EGFP expression after co-transfection of plasmid pCK1114 and the PacI-digested C101 DNA in rabbit skin cells. The C130 repair virus, C131, was created by co-transfection of the PacI-digested C130 DNA and the plasmid pCK1029 containing an EGFP expression cassette in the UL3, UL4 intergenic region and selection of EGFP positive plaques. Recombinants C132 and C134 are Δγ₁34.5 HSV-1 viruses expressing the non-immunoreactive and immunoreactive HCMV IRS1 gene PacI-digested C101 viral DNA and pCK1116 or pCK1127, respectively. The C134 repair virus, C135, was constructed by co-transfection of the PmeI-digested C134 DNA with plasmid pCK1029 and purified based on EGFP-positive plaque selection in Vero cells.

Western blotting. The antibodies used in these studies and their sources are as follows. The rabbit polyclonal antibody against phospho-eIF-2 [symbol] (p-serine-51) (44-728) and mouse monoclonal antibody against total eIF-2α(AHO0802) were purchased from Biosource International, Camarillo, Calif. Immunoblot experiments were performed as previously described, using equivalent protein mass (10 μg) loading (Cassady, K. A., et al. 1998a). In summary, nitrocellulose sheets containing the electrophoretically separated proteins were incubated in blocking solution (5% bovine serum albumin in Tris-HCl-buffered saline [TBS] containing 0.01% Tween) for at least 1 hour, reacted with antibody diluted in TBS for at least 4 hours, and then washed five times with wash buffer (TBS containing 0.1% Tween). The nitrocellulose filter was next incubated with either an appropriate alkaline phosphatase or peroxidase-conjugated antibody diluted in wash buffer for a minimum of 90 min. The filter was then washed five times with wash buffer. The alkaline phosphatase-stained immunoblots were developed using 150 μg/ml 5-bromo-4-chloro-3-indolylphosphate (BCIP) and 300 μg/ml nitroblue tetrazolium in AP buffer (100 mM Tris-HCl [pH 9.5], 5 mM MgCl2, and 100 mM NaCl), whereas the peroxidase stained immunoblots were developed using enhanced chemiluminescence as recommended by the manufacturer (Pierce, Rockford, Ill.).

DNA hybridization studies. Purification, restriction digestion, electrophoretic separation, and transfer to Zeta probe membranes (Bio-Rad, Hercules, Calif.) by capillary transfer of viral DNA has been described previously (Cassady, K. A. et al. 1998a). The separated and immobilized PstI-digested R3616, C101, C130, C131, and C132 DNAs were hybridized with a probe encoding the HSV-1 UL3 and UL4 sequences from the pRB⁴⁸⁴¹ plasmid.

PCR and RT-PCR studies. To demonstrate expression of HCMV IRS1 transcript in the C132-infected cells, reverse transcriptase (RT)-PCR was performed. Vero cells were infected with C130 and C132 at a multiplicity of infection (MOI) of 10, and total RNA was isolated from the infected cells at 18 hours post-infection (hpi) using an RNAqueous 4sure purification kit (Ambion, Austin, Tex.) per the manufacturer's instructions. The RNA was further digested with RNase-free DNase I (Ambion, Austin, Tex.) for 30 min at 37° C., followed by enzyme inactivation. First-strand synthesis was performed using SuperScript II reverse transcriptase (Invitrogen, Carlsbad, Calif.) and oligo(dT)18-21 primers on 1 μg (each) of C130 and C132 RNA. Subsequent PCR was performed on 1/10 of the first strand product using the following primers recognizing the 3′ unique domain of the IRS1 gene product: 5′ BamHI BspHI IRS263 (5′-GGATCCATCATGACAGAGCGTCAAAGTC-3′) (SEQ ID NO: 7) and 3′ HindIII IRS263 stop (5′-AAGCTTCAATGATGAACGTGGTGAGG-3′) (SEQ ID NO: 8).

UV inactivation of HCMV. HCMV was UV inactivated by exposing 3×10⁶ PFU of virus to 150 mJ of UV irradiation using a cross-linking chamber (Bio-Rad, Hercules, Calif.). Following UV irradiation, sodium pyruvate was added to a final concentration of 5 mM to neutralize any peroxide/superoxides produced during UV inactivation as described previously (Fortunato, E. A., et al. 2000).

Immunofluorescence. HFF cells were seeded on glass coverslips and mock infected or exposed to HCMV AD169 or UV-inactivated HCMV at an MOI of 3 in medium containing 10% FBS. After 2 h, the inoculum was replaced with 10% FBS. At 24 hpi, the cells were washed with phosphate-buffered saline (PBS), fixed with 4% paraformaldehyde in PBS, and processed as previously described (Sanchez, V., et al. 2000). Briefly the cells were permeabilized in 0.1% NP-40 in PBS for 10 min., washed five times with PBS, and blocked for 30 min with 10% goat serum in PBS at room temperature. After five washes with PBS, the cells were sequentially incubated for 1.5 hours with pp 65-specific monoclonal antibody (MAb) 65-8 ascites, washed with PBS, and incubated with IE1-specific (exon 4) MAb 63-27 ascites at 37° C. After being washed five times with PBS, the coverslips were incubated with goat anti-mouse immunoglobulin G1 (IgG1)-fluorescein isocyanate conjugated antibody (1:100 in PBS; Southern Biotechnology, Birmingham, Ala.) and Alexa Fluor 594 goat anti-mouse IgG2a antibody (1:400 in PBS; Molecular Probes, Eugene, Oreg.) for 1 hour at 37° C. and Hoechst for 10 minutes. Washed coverslips were mounted onto slides using SLOWFADE antiphotobleaching reagent (Molecular Probes, Eugene, Oreg.). Images were captured at ×400 magnification with an Olympus BX41 fluorescent microscope using Image-Pro Plus software (version 4.5) and processed using Adobe Photoshop 7.0.

In vivo protein synthesis. For the HCMV/HSV-1 coinfection studies, duplicate cultures of HFF cells grown in 3.8-cm2-well plates were mock infected or infected with HCMV (AD169) or UV-inactivated HCMV at an MOI of 3 PFU/cell in DMEM-1% FBS. After 2 hours, the inocula were replaced with DMEM containing 10% FBS. At 6 hours postinfection, the mock- and HCMV-infected cells were either mock infected or infected with R3616 at an MOI of 10 PFU/cell for 2 hours. At 23 hours post-HCMV infection, the cultures were incubated with 199V medium lacking methionine [199V (−) MET] but supplemented with 50 μCi of L-[35S]methionine (>1,000 Ci/mmol; Amersham-Pharmacia)/ml of media. After 1 hour of labeling, the cells were rinsed in ice-cold phosphate-buffered saline lacking Ca2+ and Mg2+ (PBS-A), scraped, resuspended in disruption buffer, boiled, and loaded on a 12% (vol/vol) polyacrylamide gel cross-linked with bis-acrylamide. The proteins were electrophoretically separated, transferred to nitrocellulose membranes, and subjected to autoradiography.

The protein labeling experiments for wild-type HSV-1 and the recombinant viruses R3616, C101, C130, C131, C132, C134, and C135 were performed as described previously (Chou, J. and B. Roizman. 1992). Briefly, HFF cells grown in 3.8-cm²-well plates were mock infected or infected with HSV-1 (F) or recombinant virus at an MOI of 10. At 14 hpi, medium was removed and replaced with 199V (−) MET supplemented with L-[35S]methionine for 1 hour. The cells were washed and lysed, and the proteins were electrophoretically separated and analyzed by autoradiography as described above.

(ii) Results

In the initial hours of infection, CMV expresses a gene product that blocked the host protein shutoff response and the phosphorylation of eIF-2α. To test the hypothesis that HCMV encoded a gene product that blocked the host protein shutoff response, coinfection experiments using either HCMV or UV-inactivated HCMV and a recombinant HSV-1 virus that triggers host protein shutoff were performed. Duplicate HFF cell cultures were either mock infected or infected with wild-type HCMV (AD169) or UV-inactivated HCMV at an MOI of 3 as described above. At 6 hpi, the mock-infected and HCMV- and UV-inactivated HCMV-infected cells were either mock infected or superinfected with an HSV-1 Δγ₁34.5 recombinant virus (R3616) at an MOI of 10 pfu/cell. At 23 hours post-initial infection (17 hours after R3616 superinfection), the cultures were metabolically labeled for the final hour of infection, washed, lysed, and solubilized in loading buffer. Ten micrograms of total protein from each of the samples was separated by sodium dodecyl sulfatepolyacrylamide gel electrophoresis. After the samples were transferred to nitrocellulose membranes, autoradiography and immunoblotting were performed.

Abundant radio-labeled proteins were detected in the mock, HCMV, and UV-inactivated HCMV singly infected cells. In the R3616 superinfected mock- and UV-HCMV-infected HFF cells, there was reduced detection of radiolabeled proteins characteristic of PKR-mediated protein shutoff and the inhibition of late viral protein synthesis. In contrast, radiolabeled proteins in the HCMV/R3616-coinfected cells, characteristic of continued HSV-1 viral protein synthesis, were detected. Therefore, HCMV infection complemented the Δγ₁34.5 virus. However, HCMV entry and delivery of virion-associated gene products were insufficient to restore wild-type protein synthesis in the R3616-infected cells.

The relative abundance of radiolabeled proteins differed dramatically between the HCMV- and the HCMV/R3616-infected cells. The presence of discrete metabolically labeled proteins was characteristic of HSV-1-infected cells and showed that HSV-1 infection limited HCMV gene expression. HSV-1 encodes several gene products, such as virion host shutoff, ICP27, that selectively enhance viral gene expression and limit cellular gene expression within 2 to 3 hours of HSV-1 infection (Hardy, W. R., et al. 1994; Kwong, A. D., and N. Frenkel. 1989; Strom, T., and N. Frenkel. 1987). Based upon the time course of the infection, the results showed that HSV-1 infection limited HCMV gene expression as early as 8 hpi. To examine whether R3616 coinfection downregulated HCMV gene expression, immunostaining experiments were performed to examine the most abundant HCMV E gene product (IE1). In comparison with the HCMV-infected cell lysate, there was reduced IE1 staining for the HCMV/HSV-1-coinfected cell lysate. As anticipated, IE1 was not detected by immunostaining in the mock-infected or UV-inactivated HCMV-infected cell samples. These data showed that HSV-1 superinfection reduced subsequent HCMV gene expression and that the complementing HCMV gene was likely expressed in the initial hours of infection, before the Δγ₁34.5 virus blocked further HCMV gene expression.

To verify that R3616 efficiently superinfected HCMV-infected cells and that the decreased IE1 production was not a global diminution in both HSV-1 and HCMV gene expression, immunoblotting with an antibody against the HSV-1 ICP0 protein was performed. Equivalent ICP0 immunostaining was observed for all of the R3616-infected samples and demonstrated that R3616 infection and gene expression proceeded independent of prior HCMV infection. As expected, ICP0 is not detected in the HSV-1-uninfected cells. Taken together, these data indicated that Δγ₁34.5 HSV-1 infection and gene expression proceed in HCMV-infected cells and that HSV-1 gene expression reduced subsequent HCMV IE1 gene expression. These data showed that the HCMV gene product that complements the Δγ₁34.5 virus was expressed during the initial hours of HCMV infection.

Experiments next examined if the late viral protein synthesis seen for HCMV/R3616-coinfected cells was from viral evasion of the PKR-mediated protein shutoff response. To test this hypothesis, immunostaining experiments to determine the relative abundance of phosphorylated eIF-2 [symbol] in the infected cell samples were performed. In uninfected cells, PKR existed in a monomeric, unactivated form. In Δγ₁34.5-infected cells, viral dsRNA triggered PKR activation, characterized by PKR dimerization, autophosphorylation, and selective phosphorylation of eIF-2α, ultimately leading to the cessation of protein synthesis. As anticipated, minimal phosphorylated-eIF-2 [symbol] in the mock-infected and UV-inactivated HCMV-exposed cell samples was detected. In contrast, phosphorylated eIF-2 [symbol] was readily detected in both the R3616-infected, mock-infected, and R3616/UV-inactivated-HCMV-coinfected cell samples. This was consistent with the autoradiograph resulted from the pulse-labeling experiment, demonstrating a reduction of protein synthesis in the infected cells. The level of phosphorylated eIF-2 [symbol] detected in the HCMV singly infected cells were similar to that in the mock-infected cell sample. These data showed that either HCMV did not trigger PKR-mediated protein shutoff in the initial 24 hours of infection or the virus encoded a protein that precluded this antiviral response. In the HCMV/R3616-coinfected cell sample, phosphorylated eIF-2 [symbol] was undetectable, indicating that HCMV prevented R3616-induced PKR-mediated protein shutoff. To verify that eIF-2αexisted in the unphosphorylated form in the HCMV singly infected and HCMV/R3616-coinfected cells, immunostaining with an antibody that detected both phosphorylated and unphosphorylated eIF-2 [symbol] was performed. The results showed that equivalent eIF-2 [symbol] was detected in all cell samples. Taken together, these data showed that a transcribed HCMV gene product, but not a virion associated gene product, complemented the Δγ₁34.5 virus and precluded Δγ₁34.5-induced host-mediated protein shutoff.

To verify that the level of UV energy used to inactivate HCMV did not damage the capsid and prevent efficient viral attachment, entry, and delivery of the virion-associated gene products, immunofluorescence studies were performed. The HCMV virion-associated protein pp 65 (pUL-83) was detectable in both the nuclei of HCMV- and UV-inactivated-HCMV-infected cells but not in mock-infected cells at 24 hpi. Consistent with the IE1 immunostaining studies, the synthesized gene product IE1 was present only in the HCMV-infected cells. Finally, Hoechst nuclear staining of the HCMV-, UV-inactivated-HCMV- and mock-infected samples demonstrated similar sample size. Taken together, these data indicated that an HCMV-transcribed gene product was required to abrogate phosphorylation of eIF-2 [symbol] and the cessation of protein synthesis induced during Δγ₁34.5 infection. In other words, human cytomegalovirus encodes a genetic mechanism expressed in the initial hours of infection to block phosphorylation of eIF-2 [symbol] and maintain protein synthesis.

Genotypes of the viruses used or derived for this study. Experiments next focused on identifying the HCMV gene that complemented the Δγ₁34.5 recombinant virus and enabled late viral protein synthesis. An HCMV gene expressed in the initial hours of infection appeared likely based upon the HSV-1 and HCMV coinfection experiments. In Δγ₁34.5-infected cells, the virus arrested at the onset of viral DNA replication and late viral gene expression. This resulted in decreased virion production and extracellular spread (Sanchez, V., et al. 2002). Two recombinant HCMV viruses, an IE2 internal deletion virus and a TRS1 deletion recombinant, had been recently described to exhibit a similar growth defect (Blankenship, C. A., and T. Shenk. 2002; Sanchez, V., et al. 2002). These HCMV genes were hypothesized to be involved in viral evasion of the PKR-mediated protein shutoff response and their absence hypothesized to lead to a delay in the transition to the late phase of HCMV infection. Initial studies suggested that the IE2 gene was not directly involved in PKR evasion but that the HCMV TRS1 gene complemented R3616 late viral protein synthesis.

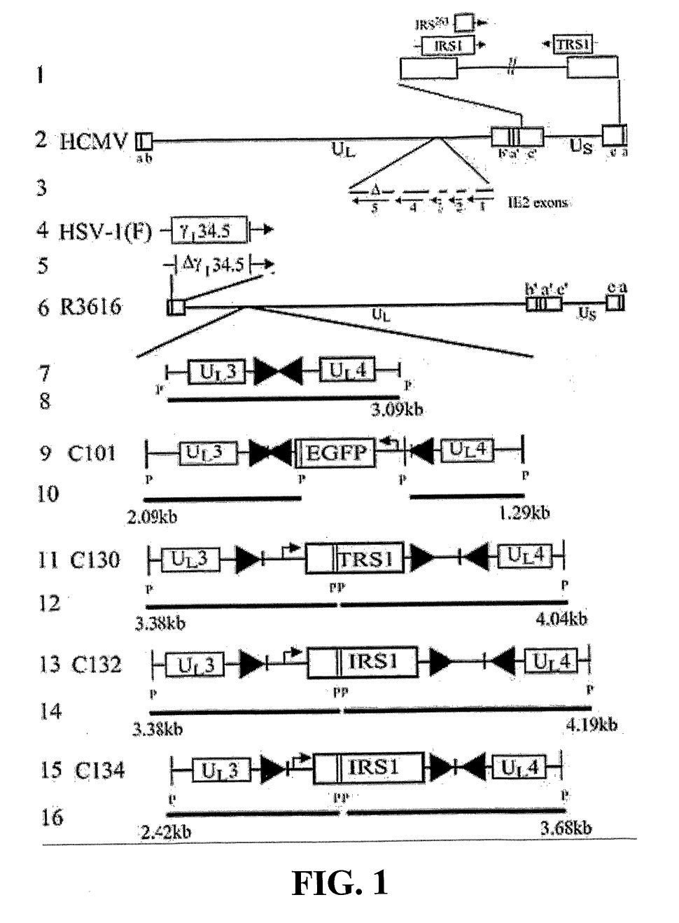

To test the hypothesis that the HCMV TRS1 gene enabled viral evasion of host protein shutoff induced by HSV-1 coinfection, Δγ₁34.5 recombinants encoding either the HCMV TRS1 or HCMV IRS1 gene products were constructed. Schematics of the recombinant viruses and the anticipated hybridization patterns for hybridization studies are presented in FIG. 1. The locations of the HCMV IRS1, TRS1, IRS263, IE2 genes, the HSV-1 Δγ₁34.5, UL3, UL4 genes, and the prototypical DNA sequence arrangements of HSV-1 and HCMV are also shown in FIG. 1.

HCMV and HSV-1 share a common genomic arrangement characteristic of class E genomes, consisting of two covalently linked long and short genetic domains, each composed of a unique domain flanked by inverted repeat domains (FIG. 1, lines 2 and 6) (Tamashiro and Spector 1986; Wadsworth, et al., 1975). The recombinant viruses constructed for this study lacked both copies of the γ₁34.5 gene, the principal HSV-1 gene involved in evasion of the PKR host protein shutoff response (FIG. 1, lines 4 and 5) (Chou, J. et al. 1990; Chou, J. and B. Roizman, 1992). The R3616 UL3, UL4 genetic domain is shown (FIG. 1, line 7). The Δγ₁34.5 recombinant virus C101 contained a 1,600-bp sequence encoding the CMV IE promoter-driven EGFP gene product flanked by the 8-bp sequence recognized by the PacI restriction enzyme in the UL3, UL4 intergenic region (FIG. 1, line 9). C101 was derived by co-transfection of R3616 viral DNA and pCK1029 plasmid DNA and sequential EGFP-positive plaque purification in Vero cells. To determine if the HCMV TRS1 or HCMV IRS1 gene precludes the shutoff of protein synthesis in Δγ₁34.5 HSV-1-infected cells, recombinant C130 (encoding the HCMV TRS1 gene product) and recombinant C132 (encoding the IRS1 gene product) were constructed (FIG. 1, lines 11 and 13). Subsequent RT-PCR and immunostaining studies showed that the IRS1 recombinant, C132, while expressing IRS1 transcript, did not make immunoreactive protein. Therefore, recombinant C134, which expressed immunoreactive IRS1 protein, was constructed. The C130-repair virus (C131) and C134-repair virus (C135) have the same genetic organization as C101 (FIG. 1, line 9).

The technique described above was used for constructing the virus. This technique enables selection of the virus on nonhuman cell lines and therefore reduces the selective pressure for secondary revertant mutations in the Δγ₁34.5 virus. To eliminate the selective advantage provided by second site mutations that restore wild-type protein synthesis, a technique initially described by Glorioso and colleagues was refined to facilitate the selection and repair of recombinant viruses rapidly without exposing the recombinant viruses to human cells (Krisky, D. M. et al. 1998). Recombinant C101 is a Δγ₁34.5 virus that encodes a colorimetric selection agent, destabilized EGFP, which was flanked by PacI restriction enzyme sites in the UL3, UL4 intergenic region of the HSV-1 genome and was instrumental in this method (FIG. 1, line 9). The 8-bp sequence recognized by the PacI restriction enzyme does not occur naturally in the 152,260 bp region of the HSV-1 genome, and its presence in the recombinant viral DNA enabled selective digestion at the intergenic region. The PacI-digested DNA was not readily replicated after transfection in mammalian cells. However, new recombinants were synthesized efficiently and selected in nonhuman cells by cotransfecting the cut viral DNA with a “rescue” plasmid carrying the new genetic information flanked by homologous domains that bridge the digested viral DNA. Recombinant Δγ₁34.5 viruses did not elicit the host-mediated protein shutoff response in Vero cells. This allowed the nonselective growth of either Δγ₁34.5 or wild-type HSV-1 viruses.

Hybridization of the electrophoretically separated, immobilized PstI digested viral DNA with a probe spanning the HSV-1 UL3 and UL4 open reading frames revealed a shift from a single 3.07-kb PstI fragment in R3616 to two PstI fragments of 2.09 and 1.29 kb in C101 created by two novel PstI sites in the EGFP cassette in C101. The 1.6-kb PstI fragment containing the EGFP open reading frame and part of the CMV promoter did not contain HSV-1 sequence and did not hybridize with the probe but was present when hybridized with an EGFP-containing probe. The recombinant C130 encoded the HCMV TRS1 gene product inserted between the UL3 and UL4 genes of Δγ₁34.5 HSV-1. The HCMV TRS1 gene contained two PstI restriction sites (21 base pairs apart) in the 5′ sequence domain. These unique restriction sites created two detectable fragments, of 4.04 kb and 3.38 kb, in the PstI-digested C130 viral DNA. The repair virus, C131, was shown to have a similar genetic organization as C101 by the DNA hybridization studies. Recombinant C132 contained the HCMV IRS1 sequence in the UL3, UL1 intergenic region. The HCMV IRS1 and TRS1 genes shared a common 5′ genetic domain but diverged in the 3′ region. Consequently, a similar hybridization pattern was seen with the C130 and C132 PstI-digested viral DNA. The C132 recombinant shares an identical 3.38-kb fragment (encoded by the UL3 and 5′ domain of the IRS1 gene) as C130; however, the unique 3′ sequence produced a slightly slower-migrating 4.19-kb fragment by Southern blotting. Recombinant C134 (expressing immunoreactive IRS1 protein) also contained a CMV IRS1 gene in the UL3, UL4 intergenic region. This recombinant was derived from a different plasmid and the cloning strategy eliminated a 960-bp sequence upstream from the HCMV IE promoter and 510 bp downstream from the polyadenylation site. The probe, therefore, hybridized with both a 2.42-kb and a 3.68-kb fragment in the C134 recombinant. The repair virus, C135, was genetically similar to C101 and C131 and also produced a 1.29-kb and a 2.09-kb restriction fragment. Immunostaining studies, using pooled IRS1 and TRS1 antisera, demonstrated that the C130 and C134 recombinant viruses produced immunoreactive TRS1 and IRS1 protein, respectively, whereas the C132 recombinant did not. Reverse transcription and PCR amplification further demonstrated that the C132 virus expressed IRS1 transcript, indicating that the recombinant contained an IRS1 gene with either a frameshift mutation or a premature stop codon. DNA contamination was not detected as demonstrated by the absence of PCR product in the RNA-alone sample.

A Δγ₁34.5 recombinant encoding the HCMV TRS1 gene product exhibited the wildtype protein synthesis phentotype. Transient expression studies showed that the HCMV TRS1 complemented Δγ₁34.5 late viral protein synthesis. To further test this hypothesis, cells infected with Δγ₁34.5 recombinant viruses expressing the TRS1 and IRS1 gene products were examined for late HSV-1 viral protein synthesis by using pulse-labeling experiments. Replicate cultures of HFF were mock infected or infected with HSV-1(F), R3616, C101, C130, C131, C132, C134, and C135 at an MOI of 10. The cultures were labeled with [³⁵S]methionine at 15 hpi for 1 hour and then processed for autoradiography as described above.

Both mock- and HSV-1(F)-infected cell samples contain abundant radiolabeled protein. The HSV-1(F)-infected cell sample contained distinct radiolabeled proteins characteristic of wild-type HSV-1 protein synthesis. In contrast, there was decreased detection of radiolabeled proteins in the R3616 and C101 infected cell samples, characteristic of the Δγ₁34.5 protein synthesis phenotype. Both of these recombinants lacked the γ₁34.5 gene and were incapable of precluding PKR-mediated protein shutoff. In the cells infected with recombinant viruses expressing the TRS1 (C130) or IRS1 (C134) protein, radiolabeled proteins were readily detectable, indicative of continued protein synthesis in the infected cell. In contrast, there was decreased detection of radiolabeled proteins in the C131, C132, and C135 and infected cells. These data indicated that expression of either the HCMV TRS1 or IRS1 protein conferred the wild-type HSV-1 protein synthesis phenotype in Δγ₁34.5-infected cells.

To verify that inhibition of PKR-mediated protein shutoff was the basis for the late viral protein synthesis witnessed with the C130- and C134-infected cells, equivalent-mass samples (10 μg), as demonstrated by equivalent actin immunostaining, were electrophoretically separated and immunostained for phosphorylated eIF-2α. The results showed that phosphorylated eIF-2 [symbol] in the R3616, C101, C131, C132 and C135-infected cell samples, characteristic of PKR-mediated protein shutoff, was readily detected. In the mock-, HSV-1(F)-, C130-, and C134-infected cell samples, phosphorylated eIF-2 [symbol] was undetectable by immunostaining and correlated with continued protein synthesis in these cells. Total eIF-2α immunostaining verified the presence of eIF-2 [symbol] in the C130 and C134 samples. These data indicated that in cells infected with a Δγ₁34.5 recombinant virus expressing either the HCMV TRS1 or IRS1 protein, eIF-2 [symbol] was maintained in the unphosphorylated state, thus allowing continued late viral protein synthesis similar to that observed with wild-type HSV-infected cells.

### Example 2

### Enhanced Anti-Glioma Activity of Chimeric Δγ₁34.5 HSV-1 Viruses Expressing HCMV Gene Products TRS1 or IRS1

(i) Materials and Methods:

Cells and viruses. U87-MG, Neuro-2a (N2A) and Vero cell lines were obtained from the American Type Culture Collection 10801 University Blvd., Manassas, Va. 20110. D54-MG and U251-MG cells were obtained from Duke University, Durham, N.C. The cells were propagated in Dulbecco modified Eagle medium (DMEM) supplemented with 5% Newborn calf serum (NBCS) (Vero) or DMEM/F12 50/50 7% Fetal Bovine Serum (FBS) (U87, D54, U251, N2A).

HSV-1 (F) and AD169 were the prototypical HSV-1 and HCMV strains, respectively. The construction of C101, C130, C131, C134, and C135 is described in Example 1 and a summary of their genetic organization is shown in FIG. 1. In brief, the C101 virus, the parent virus for all of the viruses, lacks both copies of the γ₁34.5 gene and contains the EGFP gene inserted in the UL3/UL4 intergenic region. The C130 and C134 chimeric recombinants contained the HCMV TRS1 and IRS1 genes, respectively, inserted in the UL3/UL4 intergenic region. The C131 and C135 chimeric recombinants were repair viruses that were created by replacing the TRS1 and IRS1 genes with the EGFP gene and therefore had a similar predicted genetic structure to C101. The recombinant Δ305 lacked the UL23 gene encoding the viral thymidine kinase (tk). G207 described previously, lacks the γ₁34.5 gene and contains a mutation if the viral ribonucleotide reductase gene, U_{L}39.

Protein shutoff assay—The protein labeling experiments for wild-type HSV-1, and the recombinant viruses C101, C130, C131, C134, and C135 were performed as described in Example 1. Briefly, HFF cells grown in 3.8 cm² well plates were mock infected or virus infected with HSV-1(F) or recombinant virus at an MOI of 10. At 14 hours post-infection (hpi), media was removed and replaced with 199V (−)MET supplemented with L-[³⁵S]-methionine for 1 hour (Amersham Bioscience, Piscataway, N.J.). The cells were washed, lysed and the proteins electrophoretically separated and analyzed by autoradiography.

Immunostaining of phosphorylated eIF-2 [symbol] Equivalent protein mass (10 μg) from each infected cell lysate was loaded on 12% SDS-polyacrylamide gel, electrophoretically separated, transferred to nitrocellulose and immunoblotting was performed. Antibodies used were rabbit anti-phosphorylated eIF-2α(P-serine-51) (44-728 Biosource International, Camarillo, Calif.), mouse monoclonal total eIF-2α (AHO0802 Biosource International, Camarillo, Calif.) and mouse monoclonal anti-HSV Glycoprotein D (gD) (Advanced Biotechnologies, Columbia, Md.).

Multistep replication assays—Replicate U87, D54, and U251 malignant glioma cells were infected in parallel (quadruplicate) with equivalent quantity (0.1 pfu/cell) of either wild-type, C101, C130, C131, C134, or C135 virus. Infected cell culture samples were subjected to three freeze/thaw and sonication cycles. Recovered virus was then quantified by limiting dilution assay and plaque formation in Vero cells. The experiment was repeated at least one time and the average recovered virus and standard deviation calculated for each virus and time point tested.

Survival studies—All animal studies were conducted in accordance with guidelines for animal use and care established by the University of Alabama at Birmingham Animal Resource Program and the Institutional Animal Care and Use Committee (Protocol Number 050407478). All mouse strains used were obtained from the Frederick Cancer Research and Development Center, National Cancer Institute. Mice used in survival studies were stereotactically injected intracerebrally by drilling a small hole 2 mm anteriorly and 2 mm laterally from the bregma on the right hemisphere. The needle was injected to a depth of 2.5 mm to implant tumors in the right caudate nucleus. SCID mice were injected with 1×10⁶ tumor cells (U87-MG cells), which were given fresh media in culture the day before injection, in a 5 μL volume of 5% methylcellulose. After seven days, the mice were randomly divided into cohorts, and the tumors were treated with virus administered in a 10 μL volume of PBS vehicle via the same burr hole used for cell injection. 2×10⁴ N2A cells were implanted into syngeneic A/J strain mice and virus treatments were administered after 5 days. Mice were assessed daily. Any moribund mice were killed and the date of death recorded. Survival was calculated using the Kaplan-Meier method and median survivals and 95% confidence intervals estimated (Gehan, 1972). To control for any confounding effects and to conduct stratified analyses, the Cox proportional hazards model was used. Studies were repeated at least twice to ensure biologic validity.

Neurovirulence studies—Female CBA/J strain mice (NCI) between 5 and 6 weeks of age were stereotactically injected intracerebrally with graded doses of viruses. Injections were performed as described for survival studies with viruses injected in a 10 μL volume of PBS vehicle. HSV-1(F) was used as a positive control. Mice were injected, assessed daily and deaths noted for up to 30 days. The LD50 was calculated based on Spearman-Karber statistical method.

(ii) Results

The C130 and C134 chimeric viruses maintained unphosphoylated eIF-2α and evaded PKR-mediated protein shutoff of protein synthesis in infected human glioma cells. The basis for use of Δγ₁34.5 vectors in the treatment of GBM was their selective replication in tumor cells. Alterations in the PKR cascade and protein synthesis function in the GBM tumor cells had been proposed as the basis of selective complementation and replication of Δγ₁34.5 viruses. Recent studies, however, indicated that the PKR function was intact in some malignant glioma cell lines (Shir, 2002). To test whether the Δγ₁34.5 viruses triggered host protein shutoff in malignant glioma cells and to identify the phenotype of the IRS1 and TRS1 expressing Δγ₁34.5 chimeric recombinant, the protein synthesis phenotype was assessed in infected malignant glioma cells.

Pulse labeling studies performed at late times during viral infection (14 hpi) showed that, Δγ₁34.5 viruses undergo host mediated protein shutoff in infected U87 cells. The chimeric Δγ₁34.5 recombinants, C130 and C134 behaved similar to wild-type virus in that they maintained late viral protein synthesis as demonstrated by radio-labeled protein accumulation. Inhibition of protein synthesis in the Δγ₁34.5-infected cells was a direct consequence of PKR-mediated protein shutoff, as demonstrated by the detection of phosphorylated eIF-2 [symbol] in the C101, C131, and C135 infected cell samples. Conversely, phosphorylated eIF-2 [symbol] was undetectable in the cells infected with virus exhibiting wild-type protein synthesis. These studies were repeated in both D54MG and U251MG cell lines with similar results, indicating that the chimeric recombinant viruses' evasion of PKR-mediated protein shutoff was not limited to U87MG cells.

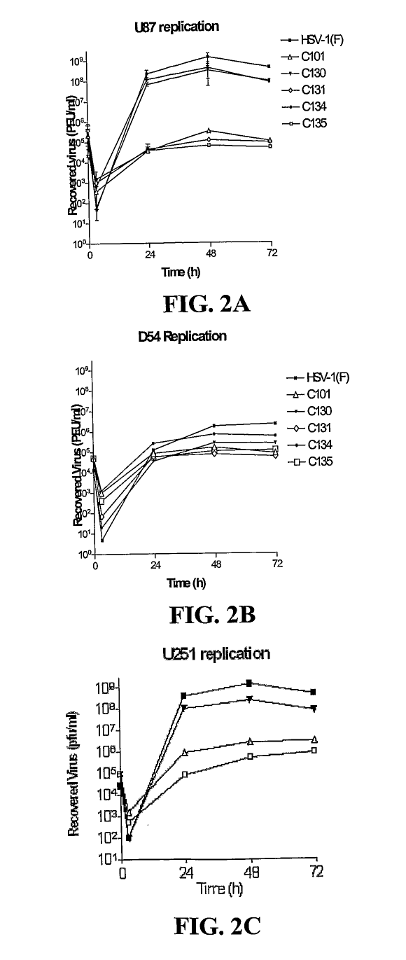

The C130 and C134 viruses replicated at near wild-type levels in U87 cells in vitro. Parallel cultures of malignant glioma U87 cells were infected with wild-type HSV-1(F) or the C101, C130, C131, C134, and C135 recombinants (0.1 pfu/cell) and virus was recovered at intervals over 3 days. The results showed that C101, C131 and C135, exhibiting a Δγ₁34.5 protein synthesis phenotype, produced 10⁴-10⁵ log virus (FIG. 2A). In contrast, the chimeric C130 and C134 infected U87 cells generated 107-101 pfu of virus, approaching that of wild-type virus (FIG. 2A). These data indicated that the viruses with a wild-type protein synthesis profile (C130, C134, and wild-type HSV-1 [F]) replicated better and generated three to four-log greater virus than recombinants with a Δγ₁34.5 phenotype in U87MG cells. The improved replication was not limited to these malignant glioma cells. The chimeric recombinants exhibited an advantage over Δγ₁34.5 recombinants and replicated at near wild-type levels in both U251MG and in D54MG cells as well (FIGS. 2B and 2C, respectively).

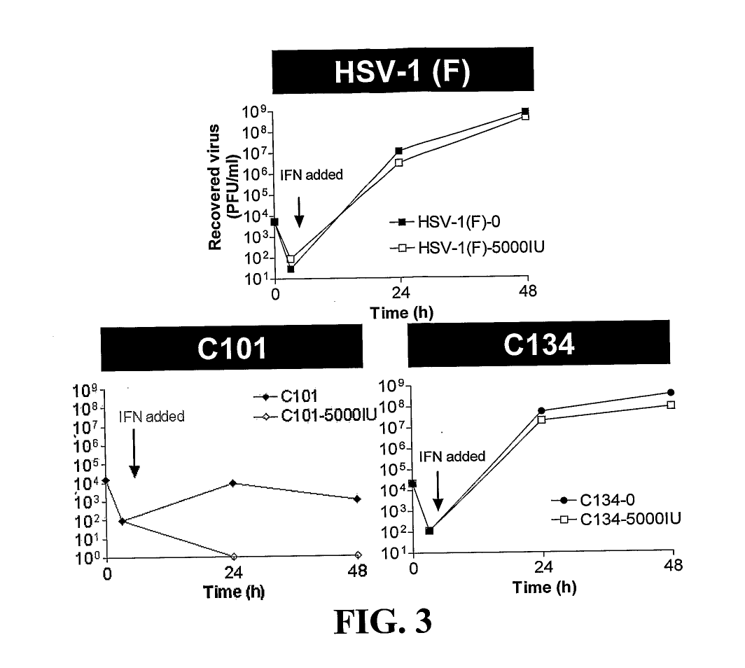

Viral replication in the presence of exogenous IFNα Low levels of PKR are present in a non-active form in unstressed cells. Its production is induced by type I interferons or dsRNA produced during viral replication. The γ₁34.5 gene encodes at least three phenotypes pertinent to anti-tumor therapy: (1) evasion of PKR-mediated host protein shutoff response, (2) Type I IFN resistance and (3) neurovirulence. Therefore, it is important for oncolytic viruses to be able to evade PKR-mediated host protein shutoff response and replicate in the presence of type I interferons without being neurovirulent. Type I interferon reduces Δγ₁34.5 virus replication. Δγ₁34.5 viruses were very sensitive to IFNα, as seen after C101 infection of U87 cells, which does not express Type 1 interferons. Expression of the HCMV genes TRS1 and IRS1 allowed high levels of viral replication in the presence of exogenous IFNα (FIG. 3). The chimeric HSV were unaffected by IFN-α treatment and generated equivalent amounts of virus in IFN treated and untreated cells. Therefore, the HCMV TRS1 and IRS1 genes restore at least two of the γ₁34.5 gene functions, viral evasion of the PKR host protein shutoff response and resistance to IFN-α. This suggests that the chimeric viruses will infect secondary tumor cells better than Δγ₁34.5 viruses in vivo.

C130 and C134 chimeric viruses exhibited neurovirulence profiles safe for administration. Since in vitro studies showed that introduction of the HCMV IRS1 or TRS1 gene into the Δγ₁34.5 recombinant produced wild-type protein synthesis and replication approaching wild-type levels in U87 cells, it was next determined whether insertion of the HCMV IRS1 and TRS1 genes into a Δγ₁34.5 recombinant also restored a wild-type neurotoxicity profile. Neurovirulence studies for both the C130 and C134 viruses were tested in six-week-old female CBA/J mice, a highly HSV-sensitive strain. All viruses were injected intracranially into the right caudate nucleus in a 10 μL volume of PBS vehicle. The LD50 values for the viruses tested calculated by Spearman-Karber analysis are summarized in Table 2. As shown, 75 pfa of wild-type virus produced a fatal encephalitis in half of the animals tested whereas the Δγ₁34.5 recombinant was aneurovirulent resulting in only a single animal death in the highest dosage group (1×10⁷ pfa). The chimeric recombinants differed in neurovirulence. The C134 recombinant demonstrated an identical safety profile as the parent virus C101 resulting in a single animal death in the highest dosage group. In contrast the C130 (TRS1, Δγ₁34.5) recombinant was more virulent than the Δγ₁34.5 virus with a calculated LD50 of 6.8×10⁵ pfu. While introduction of the TRS1 gene into HSV-1 increased the neurovirulence of the virus it did not restore wild-type neurovirulence and remained over four logs less virulent than the Δγ₁34.5-positive HSV-1 (F).

TABLE 2
Neurotoxicity of chimeric HSV-1/HCMV viruses.

| Virus | LD50 (pfu) |
|---|---|
| HSV-1 (F) | 7.5 × 10¹ |
| G207 (Δγ₁34.5, ΔU_{L}39) | >3 × 10⁸ |
| C101 (Δγ₁34.5, EGFP) | >1 × 10⁷ |
| C130 (Δγ₁34.5,TRS1) | 6.8 × 10⁵ |
| C134 (Δγ₁34.5, IRS1) | >1 × 10⁷ |
| C131 (Δγ₁34.5, C130 repair virus) | >1 × 10⁷ |

These results demonstrated the following points. First, while expression of the HCMV IRS1 or TRS1 genes by a Δγ₁34.5 HSV-1 restored one phenotype of the Δγ₁34.5 gene (wild-type protein synthesis and replication in infected tumor cells), they did not restore the other phenotype (wild-type neurovirulence). Second, while the HCMV IRS1 and TRS1 genes were interchangeable with respect to protein synthesis function and replication, viruses expressing these genes had different neurovirulence profiles. This suggested that the TRS1 and IRS1 genes encode unique functions in addition to PKR evasion. In spite of the improved replication approaching that of wild-type virus, these mutant viruses did not exhibit a concomitant wild-type neurovirulence.

Chimeric HSV reduce tumor volumes in vivo. U251-ffLuc intracranial tumors were induced in scid mice (1×10⁶ cells), and the animals were treated with a chimeric HSV (C130), a Δγ₁34.5 recombinant (R3616), or saline a week later. Luciferase activity was measured over time using IVIS® (In Vitro Imaging System) (Xenogen Corporation, Alameda, Calif.). The method involved the implantation of GBM cells stably expressing firefly luciferase enzyme and at selected times post implantation or post-virus administration, the intraperitoneal administration of a luciferase substrate (beetle luciferin, 2.5 mg/mouse) to the animal. The low molecular weight (˜1 kD) substrate, upon entering cells containing the luciferase enzyme, was cleaved into a photo-emitting chemical by an ATP-dependent process and was then rapidly degraded. The light emitted was captured digitally by a CCD camera and quantified. Because the luciferase enzyme was not present in the native animal cells and had a limited half-life at 37° C. of about 2 hours, the light emission was limited to viable, metabolically active tumor cells. The greater the number of viable GBM cells, the greater the light production.

Consistent with prior studies, the results showed that Δγ₁34.5 therapy (R3616) reduced tumor volume (based upon relative photon emission) when compared with saline treated animals, but that the chimeric HSV (C130) was more effective at reducing tumor growth (FIG. 8).

Treatment with C130 and C134 increased survival of SCID mice bearing U87 malignant glioma brain tumors. In order to evaluate the anti-tumor efficacy of the C130 and C134 viruses in vivo, SCID mice were injected stereotactically with 1×10⁶ U87 cells into the right cerebral hemisphere. Seven days after tumor induction, the mice were randomized into one of several different cohorts of virus treatments. Based on the neurovirulence studies performed above, two dose cohorts using the C130 virus were tested: one near the calculated LD50 of C130 (5×10⁵ pfa) and the second one log below (5×10⁴ pfu) lest the higher dose cause an HSV-induced encephalitis. Matching doses of the C134 virus were tested (5×10⁴ and 5×10⁵ pfu) along with a dose of 5×10⁶ pfu, since C134 was shown to have a neurotoxicity profile similar to that of the Δγ₁34.5 parent virus. As controls, 5×10⁵ pfu of the Δγ₁34.5 parent C101 virus was tested along with a vehicle (saline) treatment group. Finally, the parent Δγ₁34.5 C101 virus was also tested at the maximum tolerated dose, 1×10⁷ pfu.

TABLE 3
Anti-tumor efficacy of the chimeric HSV on survival
of the U87 human brain tumor-bearing SCID mice

| | | | p | p | p |
|---|---|---|---|---|---|
| | | Median | (vs. | (vs. C101 | (vs. C101 |
| COHORT | N | Survival | Saline) | 5 × 10⁵) | 1 × 10⁷) |
| Saline | 14 | 32.5 | | 0.0093 | <0.0001 |
| C101 5 × 10⁵ | 20 | 36.5 | 0.0093 | | <0.0001 |
| C101 1 × 10⁷ | 20 | 45 | <0.0001 | <0.0001 | |
| C130 5 × 10⁴ | 8 | Undefined | <0.0001 | <0.0001 | 0.0185 |
| C130 5 × 10⁵ | 9 | Undefined | <0.0001 | <0.0001 | 0.0004 |
| C134 5 × 10⁴ | 8 | 82 | 0.0005 | 0.0005 | 0.1498 |
| C134 5 × 10⁵ | 19 | Undefined | <0.0001 | <0.0001 | 0.0069 |
| C134 5 × 10⁶ | 11 | Undefined | <0.0001 | <0.0001 | 0.0010 |

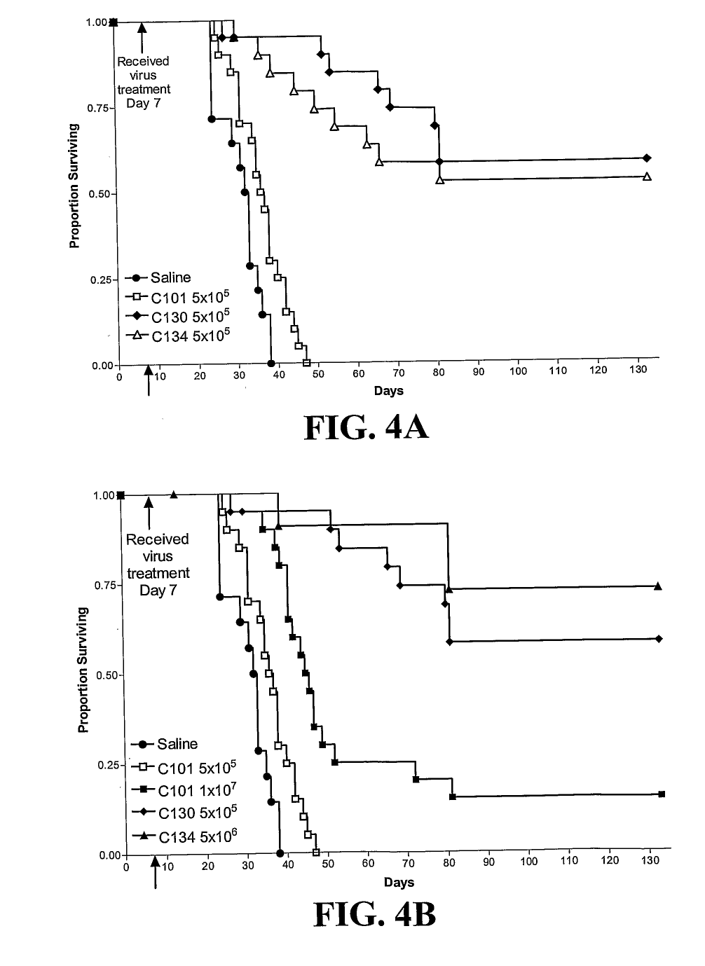

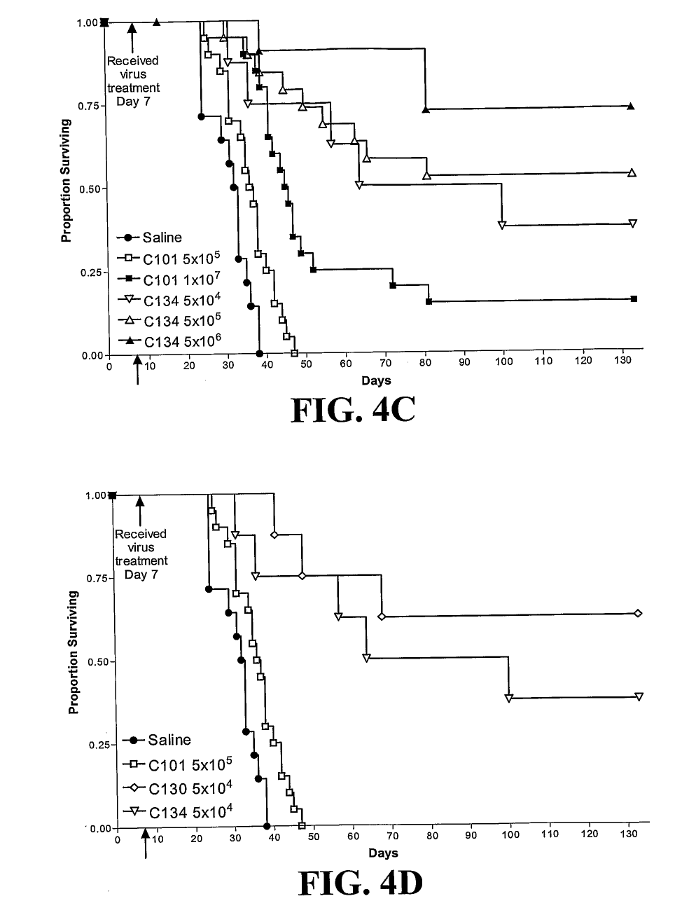

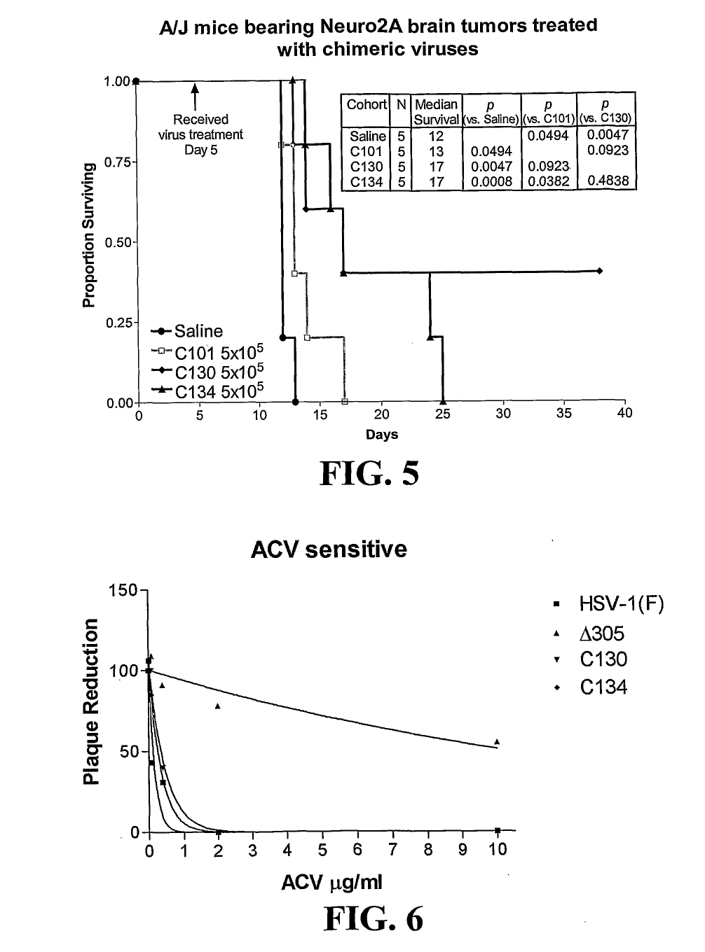

U87 human malignant gliomas (1×10⁶ cells, SCID mice) or N2A murine neuroblastomas (2×10⁴ cells, A/J mice). After one week (for U87-MG) or five days (for N2A), the brain tumor-bearing mice were treated via direct intratumoral injection of virus. The mice were then observed and deaths noted to determine survival. Control mice were treated with saline. Results are presented in FIGS. 4A to 4D, FIG. 5, Table 2 and Table 3, which show the following:

(i) The chimeric recombinant viruses were superior to the Δγ₁34.5 parent virus at equivalent doses (FIG. 4A). Both the C130 and C134 recombinant viruses produced a statistically significant improvement in survival over an equivalent dose of the Δγ₁34.5 parent virus C101 (5×10⁵ pfu, p<0.0001). While the median survival of the saline-treated mice (32.5 days) was improved with administration of 5×10⁵ pfa of the oncolytic virus C101 (36.5 days; saline vs. C101 5×10⁵ pfu, p=0.0093), ultimately all of the animals died (FIGS. 4A to 4D). In contrast, the majority of the animals treated with 5×1 dose of either the C130 or C134 chimeric virus survived, such that a median survival could not be determined (C101 vs. C130 or C134, 5×10⁵ pfu, p<0.0001).

(ii). The chimeric recombinant viruses demonstrated superior anti-tumor activity to the C101 recombinant after administration of the maximum tolerated doses (MTD) (FIG. 4B). An antiglioma therapeutic would optimally be administered at the maximum tolerated dose. The chimeric recombinant viruses, C130 and C134, were then compared against the maximum administrable dose of the C101 recombinant, 1×10⁷ pfu. The TRS1 and IRS1 expressing recombinant viruses, at a dose of 5×10⁵ and 5×10⁶ pfa, respectively, produced a statistically significant improvement in survival over the MTD (1×10⁷) of the C101 recombinant (C101 1×10⁷ vs. C130 5×10⁵, p=0.0004; C101 10×10⁷ vs. C134 5×10⁶, p=0.0010) (Table 3). At these doses, while 20% of the C101 treated animals survived, the majority of the C130 (73%) and C134 (60%) survived.

(iii). The survival of both C101—and chimeric recombinant-treated animals improved with dose escalation (FIG. 4C). Although the chimeric and C101 recombinants were selectively replication-competent viruses and should theoretically be capable of disseminating throughout the tumor mass after inoculation, administration of a higher initial dose of virus (C101 1×10⁷ pfu) significantly increased the survival of the mice (median survival 45 days; p<0.0001 for C101 5×10⁵ vs. C101 1×10⁷) (Table 3). Administration of a higher dose was possible because of the safe neuropathogenic profile of the virus. A similar trend was seen for treatment with C134. Escalating initial treatment doses improved survival time and increased the percentage of long-term survivors (9C).

(iv). Low doses of the chimeric recombinants demonstrated a superior anti-tumor efficacy over the highest tested dose of C101. Even when using a two to three log lower dose of the C130 or C134 recombinants (5×10⁴ and 5×10⁵ pfu), the HSV-1 vectors expressing TRS1 or IRS1 still outperformed the 1×10⁷ pfu dose of the C101 virus. The C130 recombinant produced a statistically significant improvement in survival when compared with the highest dose of C101 tested (C101 1×10⁷ compared to C130 5×10⁴ pfu, p=0.0185) and the majority of animals treated with 5×10⁴ pfu dose of the C130 recombinant survived (FIG. 4D and Table 3). However, the safer C134 chimeric was clearly superior to the MTD of the Δγ₁34.5 recombinant when administered at the 5×10⁵ dose (p=0.0069) (FIG. 4C). The C134 recombinant, although exhibiting a trend toward superiority in median survival did not produce a statistically significant improvement in survival over the maximum tolerated dose of C101 when administered at the 5×10⁴ dose (C101 1×10⁷ median survival 46 days vs. C134 5×10⁴ median survival 82 days, p=0.1498) (Table 3).

(v). The chimeric recombinants exhibited a similar antiglioma effect, independent of their neurovirulence profile. Thus, these survival studies demonstrated that Δγ₁34.5 HSV-1 chimeric vectors expressing the TRS1 or IRS1 genes significantly improved the survival of U87 tumor-bearing SCID mice compared to treatment with a Δγ₁34.5 parent virus. This benefit was evident even when two to three log lower doses of C130 or C134 were used. The recombinant viruses tested were more effective (prolonging survival) at higher doses despite being replication-competent viruses. Finally, although the TRS1- and IRS1-expressing HSV-1 vectors differed in their neurovirulence profile, their anti-tumor efficacy profile was similar.

Treatment with C130 and C134 increased survival of mice bearing syngeneic murine neuroblastoina N2A brain tumors. The antiglioma benefit of the TRS1- and IRS1-expressing C130 and C134 viruses extended to other tumor types as well. The Neuro-2a murine neuroblastoma model provided a stringent test for chimeric HSV oncolytic activity because Neuro-2A cells do not support efficient HSV infection and elicit no discernible anti-tumor immune response. CBA/J mice bearing the syngeneic murine neuroblastoma N2A brain tumors also demonstrated improved survival after treatment with chimerics C130 and C134 (FIG. 5).

The C130 and C134 viruses remained susceptible to acyclovir. Any HSV-1 vector to be used clinically as an anti-tumor agent should not only possess an acceptable neurovirulence profile but also retain sensitivity to anti-viral agents such as acyclovir. In vitro plaque reduction assays on the Δγ₁34.5 chimeric C130 and C134 viruses retained their susceptibility to acyclovir in contrast to the thymidine kinase (tk)-negative Δ305 virus (FIG. 6). Therefore, these viruses remain unique from most non-HSV vectors in that standard anti-viral drug regimens would still be available for eliminating any unrestrained infections, though the neurovirulence studies suggested this to be an unlikely event.

Improved glycoprotein D (gD) expression correlates with improved protein synthesis. Viruses with the wild-type protein synthesis phenotype accumulate more glycoprotein D. In this way gD can act as a surrogate marker for protein synthesis phenotype. Glycoprotein D immunostaining from 4TI murine breast cancer and gD immunostaining from Human U87-MG cells infected was determined for various recombinant viruses. Cells were infected at a high multiplicity of infection (10 plaque forming units/cell), harvested at 18 hours post-infection, washed with PBS, boiled in SDS containing disruption buffer and the proteins separated by SDS-PAGE. There is greater detection of gD in the wild-type, C130 (TRS chimeric) and C134 (IRS chimeric) infected samples indicative of prolonged viral protein synthesis. In cells infected with viruses exhibiting a Δγ₁34.5 protein synthesis phenotype, while gD is detectable, there is decreased accumulation of the glycoprotein. These results demonstrate that C130 and C134 chimeric viruses have improved viral protein synthesis in different tumor cell types, which should result in improved viral replication and oncolysis.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,”, “an,” and “the” include plural reference unless the context clearly dictates otherwise. Thus, for example, reference to “a virus” includes a plurality of such viruses, reference to “the virus” is a reference to one or more viruses and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material or methods for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### REFERENCES

Abraham, et al., (1998). Exp. Cell Res. 244(2): 394-404.
Adamo, et al., (2004). J. Virol. 78:10221-10229.
Advani, et al., (1999) Cancer Res. 59(9):2055-8.
Advani, et al., (1998) Gene Therapy 5(2):160-165.
Andreansky, et al., (1998). Gene Ther. 5(1): 121-30.
Andreansky, et al., (1997). Cancer Res. 57(8): 1502-9.
Bennett, et al., (2001). Ann. Surg. 233(6): 819-26.
Black, et al., (1993). J. Virol. 67:791-800.
Blankenship and Shenk. (2002). J. Virol. 76:12290-12299.
Boyle, et al., (1999). Mol. Cell. Biol. 19:3607-3613.
Burysek and Pitha., (2001). J. Virol. 75:2345-2352.
Carroll, et al., (1993). J. Biol. Chem. 268:12837-12842.
Cassady, (2005). J. Virol. 79(14): 8707-15.
Cassady, et al., (1998). J. Virol. 72:8620-8626.
Cassady, et al., (1998). J. Virol. 72:7005-7011.
Cassady, et al., (2002). J. Virol. 76(3): 942-9.
Chang, et al., (2002). Neuroreport 13(18): 2429-32.
Chen, et al., (2005). J. Neurosurg. 103(2):311-319.
Cheng, et al., (2003). J. Virol. 77(18): 10154-61.
Child, et al., (2004). J. Virol. 78:197-205.
Child, et al., (2002). J. Virol. 76:4912-4918.
Chiocca, et al., (2003). Cancer J. 9(3): 167-79.
Chou and Roizman, (1992). Proc. Natl. Acad. Sci. USA 89:3266-3270.
Chou, et al., (1990). Science 250(4985): 1262-6.
Chou, et al., (1995). Proc. Natl. Acad. Sci. USA 92(23): 10516-20.
Chung, et al., (2002). Gene Therapy 9(1):75-80.
Clemens and Bommer (1999). Int. J. Biochem. Cell Biol. 31(1): 1-23.
Cobbs and Markert (1999). “Gene Therapy of Glioma: A Review.” Perspectives in Neurological Surgery: in press.
Cunningham, et al., (2000). Cell Transplant 9(5): 585-94.
Davies, et al., (1993). J. Virol. 67:1688-1692.
Duncan and Hershey. (1984). J. Biol. Chem. 259:11882-11889.
Ejercito, et al., (1968). J. Gen. Virol. 2:357-364.
Farassati, et al., (2001). Nat. Cell Biol. 3(8): 745-50.
Fortunato, et al., (2000). Proc. Natl. Acad. Sci. USA 97:853-858.
Franklin and Johnson (1998). J. Cell Biol. 142(5): 1313-24.
Fu, et al., (2003). Mol. Ther. 7(6): 748-54.
Gehan, (1972). Biometrics 28(1): 239-44.
Hardy and Sandri-Goldin. (1994). J. Virol. 68:7790-7799.
He, et al., (1997). Proc. Natl. Acad. Sci. USA 94:843-848.
Hellums, et al., (2005). Neuro-oncol. 7(3): 213-24.
Hii, et al., (2004). Int. J. Cancer 109(3):329-35.
Hosli, et al., (1998). Annals of Oncology 9(6): 589-600.
Iskenderian, et al., (1996). J. Virol. 70:383-392.
Jagus, et al., (1999). Int. J. Biochem. Cell Biol. 31(1): 123-38.
Katze, (1995). Trends Microbiol. 3(2): 75-8.
Kawaguchi, et al., (1997). J. Virol. 71:7328-7336.
Kim, et al., (2002). Oncogene 21(57): 8741-8.
Krisky, et al., (1998). Gene Ther. 5:1517-1530.
Kumar, et al., (1997). EMBO J. 16:406-416.
Kuriyama, et al., (2001). Cancer Res. 61(5): 1805-9.
Kuriyama, et al., (1998). Anticancer Res. 18(5A):35 3399-406.
Kwong and Frenkel. (1989). J. Virol. 63:4834-4839.
Lasner, et al., (1998). J. Neurovirol. 4(1): 100-5.
Lee, et al., (1994). Proc. Natl. Acad. Sci. USA 91:10551-10555.
Lee, et al., (1994). Mol. Cell. Biol. 14:2331-2342.
Leopardi and Roizman (1996). Proc. Natl. Acad. Sci. USA 93(18): 9583-7.
Link, (1984). Biometrics 40(3): 601-9.
Lockshin and Zakeri, (2004). Int. J. Biochem. Cell Biol. 36(12): 2405-19.
Lu, et al., (1995). Virology 214:222-228.
Markert, et al., (2000). Rev. Med. Virol. 10(1): 17-30.
Markert, et al., (2000). Gene Ther. 7(10): 867-74.
Mathews and Shenk, (1991). J. Virol. 65:5657-5662. 8714.
Mezhir, et al., (2005). Cancer Res. 65(20):9479-9484.
Miller and Fraser, (2000). Cancer Res. 60(20): 5714-22.
Mizushima, (2004). Int. J. Biochem. Cell Biol. 36(12): 2491-502.
Mohr and Gluzman, (1996). EMBO J. 15:4759-4766.
Mohr, et al., (2001). J. Virol. 75(11): 5189-96.
Montine and Henshaw, (1989). Biochim. Biophys. Acta 1014:282-288.
Nussbaum, et al., (2003). Cancer Lett. 196(2): 207-16.
Ortega, et al., (1996). Virology 215(1): 31-9.
Pandita, et al., (2004). Genes Chromosomes Cancer 39(1): 29-36.
Parker, et al., (2000). Proc. Natl. Acad. Sci. USA 97(5): 2208-13.
Parker, et al., (2005). Cancer Gene Ther. 12(4): 359-68.
Plachter, et al., (1993). Virology 193:642-652.
Post and Roizman, (1981). Cell 25:227-232.
Pritchett (1980). J. Virol. 36:152-161.
Rainov, et al., (1996). Cancer Gene Ther. 3(2): 99-106.
Rampling, et al., (2000). Gene Ther. 7(10): 859-66.
Romanowski and Shenk. (1997). J. Virol. 71:1485-1496.
Romanowski, et al., (1997 J. Virol. 71:5703-5705.
Saifi, et al., (2005). Hum. Mutat. 25(4): 372-83.
Samuel (2001). Clin. Microbiol. Rev. 14(4): 778-809 (Table of Contents)
Sanchez, et al., (2002). J. Virol. 76:2973-2989.
Sanchez, et al., (2000). J. Virol. 74:975-986.
Shah, et al., (2003). J. Neurooncol. 65(3): 203-26.
Sharp, et al., (1993). Nucleic Acids Res. 21: 4483-4490.
Shir and Levitzki (2002). Nat. Biotechnol. 20(9): 895-900.
Shirk, et al., (2005) J. Neurosci. Res. 82(1): 43-50.
Smith, et al., (2006) J. Virol. 80(3): 1110-1120.
Stasiak and Mocarski, (1992). J. Virol. 66:1050-1058.
Strom and Frenkel. (1987). J. Virol. 61:2198-2207.
Sudhakar, et al., (2000). Biochemistry 39:12929-12938.
Talloczy, et al., (2002). Proc. Natl. Acad. Sci. USA 99(1): 190-5.
Tamashiro and Spector, (1986). J. Virol. 59:591-604.
Tanaka, et al., (1998). Cancer Research 58(15): 3362-3369.
Thomis and Samuel (1993). J. Virol. 67(12): 7695-700.
Voges, et al., (2003). Ann. Neurol. 54(4): 479-87.
Wadsworth, et al., (1975). J. Virol. 15:1487-1497.
Wakimoto, et al., (2004). Gene Ther. 11(2): 214-23.
Williams, (1999). Oncogene 18(45):6112-20.
Williams, (2001). Sci. STKE 2001 (89): RE2.
Williams, et al., (2003). Antimicrob. Agents Chemother. 47:2186-2192.
Zhan, et al., (1994). Mol. Cell. Biol. 14:2361-2371.
Zhou and Roizman (2005). J. Virol. 79(9): 5272-7.
Zhou, et al., (2002). Proc. Natl. Acad. Sci. USA 99(23): 15124-9.
Zhu, et al., (1997). Proc. Natl. Acad. Sci. USA 94:13985-13990.

What is claimed is:

1. A chimeric virus comprising:
a. a modified herpesvirus nucleic acid sequence, wherein the herpesvirus nucleic acid modification causes reduced expression of a protein kinase R (PKR) evasion gene as compared to expression of the evasion gene in the absence of the modification; and
b. a second viral nucleic acid sequence, wherein the second viral sequence encodes a protein that inhibits or compensates for at least one activity of PKR.

2. The chimeric virus of claim 1, wherein the modified herpesvirus nucleic acid is a modified α herpesvirus virus nucleic acid.

3. The chimeric virus of claim 2, wherein the modified herpesvirus nucleic acid is a modified HSV-1 nucleic acid.

4. The chimeric virus of claim 2, wherein the modified herpesvirus nucleic acid is a modified HSV-2 nucleic acid.

5. The chimeric virus of claim 1, wherein the modified herpesvirus nucleic acid is a β herpesvirus virus nucleic acid.

6. The chimeric virus of claim 1, wherein the modified herpesvirus nucleic acid is a γ herpesvirus virus nucleic acid.

7. The chimeric virus of claim 1, wherein the modified herpesvirus nucleic acid sequence comprises a deletion or mutation of a gamma (1)34.5 gene (γ₁34.5) or a nucleic acid with at least about 70% homology to the γ₁34.5 gene.

8. The chimeric virus of claim 1, wherein the modified herpesvirus nucleic acid sequence comprises an exogenous stop codon or an exogenous promoter that alters expression of a gamma(1)34.5 gene (γ₁34.5) or a nucleic acid with at least about 70% homology to the γ₁34.5 gene.

9. The chimeric virus of claim 1, wherein the second viral nucleic acid sequence is a cytomegalovirus (CMV) nucleic acid.

10. The chimeric virus of claim 9, wherein the CMV nucleic acid comprises a IRS-1 gene or a nucleic acid having at least about 70% homology to the IRS-1 gene.

11. The chimeric virus of claim 9, wherein the CMV nucleic acid comprises a TRS-1 gene or a nucleic acid having at least about 70% homology to the TRS-1 gene.

12. The chimeric virus of claim 1, wherein the virus has reduced neurovirulence as compared to a wild-type herpesvirus virus.

13. The chimeric virus of claim 1, wherein the second nucleic acid enhances protein synthesis or replication as compared to the protein synthesis or replication of the chimeric virus in the absence of the second viral nucleic acid sequence.

14. A method of selectively killing a target cell wherein the cell is contacted with the chimeric virus of claim 1.

15. The method of claim 14, wherein the target cell is a cancer cell.

16. The method of claim 15 wherein the cancer cell is selected from the group consisting of an adenocarcinoma, hepatoblastoma, sarcoma, glioma, glioblastoma, neuroblastoma, plasmacytoma, histiocytoma, melanoma, adenoma, myeloma, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, and pancreatic cancer cell.

17. The method of claim 15, wherein the cancer cell is a solid tumor cell.

18. The method of claim 17, wherein the cancer cell is a neuroblastoma cell.

19. The method of claim 17, wherein the cancer cell is a glioma cell.

20. The method of claim 17, wherein the cancer cell is a breast cancer cell.

21. A method of treating cancer in a subject comprising contacting a cancer cell with the chimeric virus of claim 1.

22. The method of claim 21, wherein the cancer is selected from the group consisting of adenocarcinoma, sarcoma, glioma, glioblastoma, neuroblastoma, plasmacytoma, a, bladder cancer, brain cancer, squamous cell carcinoma of the head and neck, ovarian cancer, skin cancer, liver cancer, lung cancer, colon cancer, cervical cancer, breast cancer, renal cancer, esophageal carcinoma, head and neck carcinoma, testicular cancer, colorectal cancer, prostatic cancer, and pancreatic cancer.

23. The method of claim 21, wherein the cancer is a glioblastoma.

24. The method of claim 21, wherein the cancer is a neuroblastoma.

25. The method of claim 21, wherein the cancer is a breast cancer.

26. The method of claim 21, further comprising administering to the subject a chemotherapeutic agent.

27. A viral vector comprising the chimeric virus of claim 1, wherein the chimeric virus further comprises an exogenous gene of interest.

28. The viral vector of claim 27 wherein the gene of interest encodes HIV-1 GAG, IL-12, GM-CSF, IL-15, CCL2, IL-18, IL-24, IL-4, IL-10 TNF-α, purine nucleoside phosphorylase (PNP) or cytosine deaminase (CD).

29. The viral vector of claim 27 wherein the gene of interest encodes IL-12.

30. The vector of claim 27, wherein the gene of interest encodes a therapeutic agent.

31. The vector of claim 30 wherein the therapeutic agent is a chemotherapeutic agent.

32. The vector of claim 26, wherein the gene of interest encodes a targeting moiety.

33. A method of delivering a gene of interest to a cell, comprising contacting the cell with the viral vector of claim 27.
